(19) United States
(12) Patent Application Publication
(10) Pub. No.: US 2007/0157342 A1
(43) Pub. Date: Jul. 5, 2007

(54) Methods and Compositions for Increasing The Nitrogen Storage Capacity of A Plant

(75) Inventors: Kanwarpal S. Dhugga, Johnston, IA (US); Laura M. Appenzeller, Adel, IA (US); Rajeev Gupta, Johnston, IA (US); Hari Kishan Rao Abbaraju, Johnston, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL, INC., Johnston, IA (US)

(21) Appl. No.: 11/611,911

(22) Filed: Dec. 18, 2006

Related U.S. Application Data
(60) Provisional application No. 60/751,871, filed on Dec. 20, 2005.

(51) Int. Cl. A01H 5/00; C12N 15/82; C12N 5/04

(52) U.S. Cl. 800/287; 800/320; 800/320.1; 800/320.2; 800/320.3; 435/412; 435/468

(57) ABSTRACT

The present invention provides methods and compositions for making and using transgenic plants that exhibit increased nitrogen storage capacity compared to wild-type plants. Methods of the invention comprise inducing overexpression of monocot-derived vegetative storage proteins (VSPs) in plants, particularly in monocots. In some embodiments, at least one nucleotide construct comprising a nucleotide sequence encoding the ZmLox6 protein or a biologically active fragment or variant thereof is introduced into a plant. Depending upon the objective, the nucleotide construct may optionally comprise an operably linked coding sequence for a vacuolar sorting signal or plastid transit peptide in order to direct storage of the ZmLox6 protein or biologically active fragment or variant thereof into the vacuolar compartment or plastid compartment, respectively, of the cells in which the VSP is expressed. The invention further provides methods for producing plants with increased nitrogen content and/or increased nutritional value, which is desirable in commercial crops, including those used for forage, silage, and grain production.

### CROSS REFERENCE

This utility application claims the benefit U. S. Provisional Application No. 60/751,871, filed Dec. 20, 2005, which is incorporated herein by reference.

### FIELD OF THE INVENTION

The invention relates to the field of biochemistry and molecular biology. More specifically, this invention pertains to increased nitrogen storage capacity in a plant conferred by expression of a vegetative storage protein.

### BACKGROUND OF THE INVENTION

The global demand for nitrogen fertilizer for agricultural production currently stands at about 90 million metric tons per year, and is projected to increase to approximately 240 million metric tons by the year 2050. A substantial amount of nitrogen applied during crop production is lost by leaching and denitrification, which not only adds to the cost of agricultural production but contributes to environmental pollution. For example, leached nitrate pollutes groundwater, while runoff water from nitrogen-rich farmland causes algal growth in rivers and deltas. Excess nitrogen in groundwater and runoff water can also cause health problems in humans and livestock due to high intake of nitrogen in its nitrate form.

A number of crop production techniques have been proposed to reduce nitrogen losses from crop fields. Agricultural best management practices have focused on reducing the amount of nitrogen leaving agricultural fields by improving nitrogen application techniques, employing alternative cropping systems, and use of improved drainage methods. However, such practices have typically suffered from low compliance among farmers, due in part to a lack of appropriate incentives. Although public wastewater treatment plants decrease nitrogen content in part by converting nitrate into ammonia, additional treatment to remove nitrate is uncommon due to high associated costs. Natural wetlands have also been used for nutrient removal at a lower cost and greater effectiveness compared to conventional treatment plants, but such use has caused unintended biological consequences like selective growth of some plant species.

One alternative to the methods described above is to develop new crop varieties that are more efficient in absorbing and utilizing nitrogen from the soil. Many plants are known to sequester excess nitrogen in their vegetative cells by accumulating a class of proteins referred to as vegetative storage proteins (VSPs). VSPs range in size from about 15 to about 100 kDa, and have been identified from other classes of proteins such as alkaline phosphatases, chitinases, lectins, and lipoxygenases. The occurrence of VSPs has been reported in a wide variety of annual and perennial plant species including soybean, clover, alfalfa, Medicago, Arabidopsis, canola, poplar, black mulberry, and peach. However, the occurrence of VSPs in monocots has not heretofore been established.

Thus, the present invention solves needs for increasing the nitrogen storage capacity of plants, particularly in monocots, by increasing the expression of monocot-derived VSPs.

### BRIEF SUMMARY OF THE INVENTION

Methods and compositions are provided for increasing the nitrogen storage capacity of a plant, particularly within vegetative cells of the plant. The methods of the invention comprise increasing the expression of vegetative storage proteins (VSPs) within the cells of a plant, particularly expression of a monocot-derived VSP or biologically active fragment or variant thereof that has VSP properties. In this manner, the methods comprise introducing into a plant of interest at least one nucleotide construct comprising a polynucleotide sequence that includes a coding sequence for a monocot-derived VSP or a biologically active fragment or variant thereof, where the coding sequence is operably linked to a promoter that drives expression in a plant cell. In some embodiments, the VSP is the maize VSP-type lipoxygenase ZmLox6 protein set forth in SEQ ID NO: 2, and the nucleotide construct comprises the coding sequence for ZmLox6 as set forth in nucleotides 62-2737 of SEQ ID NO: 1 or in SEQ ID NO: 3, a nucleotide sequence encoding the ZmLox6 protein, or a nucleotide sequence encoding a biologically active fragment or variant of the ZmLox6 protein. Depending upon the desired subcellular localization for sequestration of the VSP, the nucleotide construct can optionally comprise a coding sequence for a vacuolar sorting signal or plastid transit peptide to direct storage of the VSP or fragment or variant thereof into the vacuolar or plastid compartment, respectively, of the plant cells in which the VSP or fragment or variant thereof is expressed. Any functional promoter can be used to drive expression of the VSP or fragment or variant thereof, with or without the vacuolar sorting signal or plastid transit peptide, including but not limited to constitutive, inducible, and tissue-preferred promoters. In some embodiments, the operably linked promoter is a leaf-preferred promoter so that levels of VSP, more particularly ZmLox6 or fragment or variant thereof, are increased preferentially within the leaf tissues of the plant. The promoter can optionally be chosen to provide for expression of the VSP or fragment or variant thereof in a cell-preferred manner, for example, a mesophyll cell-preferred or bundle-sheath cell-preferred manner, to minimize impact of VSP accumulation on cellular metabolic processes.

By increasing nitrogen storage capacity within cells of a plant, overall plant responsiveness to applied soil nitrogen can be increased, leading to improved utilization of available soil nitrogen. The methods of the invention also provide for increasing nitrogen content of a plant, particularly within the leaf, stem, and seed tissues, which beneficially increases the nutritional value of forage and silage crop plants, as well as the nutritional value of seed, particularly grain of agricultural crop species.

Compositions of the invention include nucleotide constructs comprising operably linked coding sequences for a vacuolar sorting signal and the maize ZmLox6 VSP or a biologically active fragment or variant thereof having VSP properties, and an operably linked promoter. The operably linked promoter can be any promoter that drives expression in a plant cell, including but not limited to a constitutive, inducible, or tissue-preferred promoter. Further provided are plants, plant cells, plant tissues, and transgenic seeds comprising these nucleotide constructs. These constructs find use in the methods of the invention to enhance nitrogen storage capacity of vegetative plant cells, to increase nitrogen content of a plant or plant part thereof, to increase nutritional value of forage and silage crop plants, and to increase nutritional value of seed.

### BRIEF DESCRIPTION OF THE DRAWINGS

FIG. 1 shows vectors carrying the ZmLox6 coding sequence under the control of a Rubisco small subunit (SSU) promoter. FIG. 1A shows a vector without the operably linked coding sequence for the Zea mays (ZM) proaleurain signal peptide (SP) and vacuolar sorting signal (VTS). FIG. 1B shows a vector with the operably linked coding sequence for the ZM proaleurain SP and VTS.

FIG. 2 shows vectors carrying the ZmLox6 coding sequence under the control of the Zea mays (ZM) phosphoenolpyruvate carboxylase (PEPC1) promoter. FIG. 2A shows a vector without the operably linked coding sequence for the ZM proaleurain SP and VTS. FIG. 2B shows a vector with the operably linked coding sequence for the ZM proaleurain SP and VTS.

FIG. 3 shows vectors carrying the ZmLox6 coding sequence under the control of the constitutive Zea mays (ZM) UBI promoter. FIG. 3A shows a vector without the operably linked coding sequence for the ZM proaleurain SP and VTS. FIG. 3B shows a vector with the operably linked coding sequence for the ZM proaleurain SP and VTS.

FIG. 4 shows SDS-PAGE results for the soluble fraction of homogenates from different tissues taken from plants grown in the presence of four different nitrogen levels. Three sets of four columns are shown, corresponding to the soluble fraction of homogenates from leaf (left-hand set), root (middle set), and stem (right-hand set). Within each set, the four columns correspond to homogenates from plants grown in the presence of either no nitrate (“0”), 1 mM nitrate (“1”), 100 mM nitrate (“100”), or a combination of 50 mM ammonium and 50 mM nitrate (“50+50”). The arrow in the leaf set points to an ˜100 kDa polypeptide band identified in leaf tissue at higher levels of nitrogen exposure.

FIG. 5 shows twelve different peptide sequences identified following excision of the ˜100 kDa polypeptide band shown in FIG. 4, digestion, and sequencing of collected proteolytic peptides. As shown, these peptides correspond to various segments of the ZmLox6 polypeptide (SEQ ID NO: 2).

FIG. 6 shows a phylogenetic comparison of ZmLox6 to Lox proteins from maize and other plant species.

FIG. 7 shows a sequence alignment of the ZmLox6 (SEQ ID NO: 2) and ZmLox10 (SEQ ID NO: 4) polypeptides using Vector NTI. Conserved regions are shaded, with exact residue matches shown in grey text.

FIG. 8 shows a graph comparing the induction of expression of the ZmLox6 gene in the V5 corn leaf at V5 stage of development following wounding. Induction of expression (measured in ppm) is shown over time at 0, 3, 12 and 24 hours following wounding.

FIG. 9 shows a graph comparing the induction of expression of the ZmLox6 gene in the corn nodal root at V5 stage of development following wounding. Induction of expression (measured in ppm) is shown over time at 0, 3, 12 and 24 hours following wounding (“W” group), as compared to unwounded experimental controls (“U” group).

FIG. 10 shows the expression levels of ZmLox10 in the leaves of B73, ILP, and IHP.

FIG. 11 shows the expression and purification of the ZmLox6 protein in the vector pET28A in Rosetta cells. Notice high level of expression of the protein at ˜100 kDa.

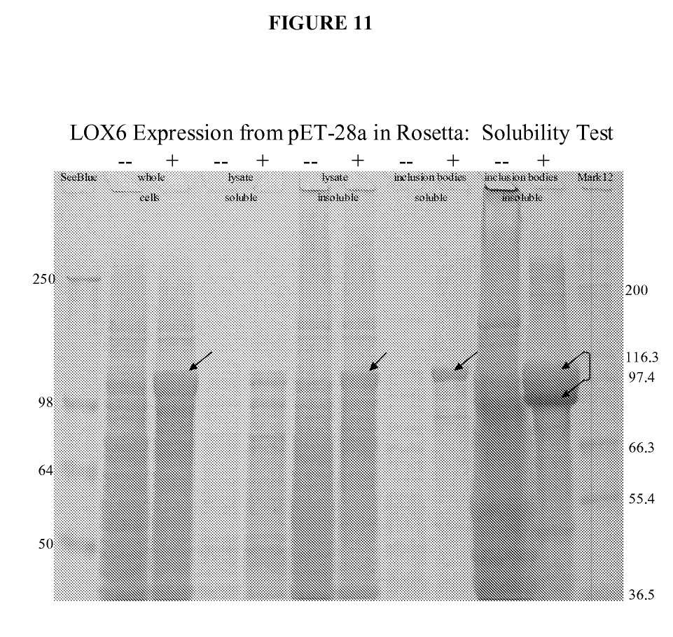

FIGS. 12A and 12B show the SDS gels (left) and a corresponding Western blot (right) of different leaf sections, and vascular bundles and mesophyll cells derived from the leaf sheath. Notice expression of the ZmLox6 protein mainly in the mesophyll cells.

FIG. 13: Titration of anti-Lox6 antibody for ELISA assay development. Titrating for antibody dilution is given for the Lox6 protein where the absorbance was linear from 1:15,000 to 1:40,000 dilutions

FIG. 14: Expression of Lox6 protein in maize leaves. Transgenic plants from the To generation expressing the ZmLox6 gene. Multiple transgenic events were obtained from six different constructs (for vector construction information, refer to FIG. 2). Abbreviations: Ubi-Intron, maize ubiquitin promoter along with a piece of an intron; PEPC, maize phoshpoenolpyruvate carboxylase promoter; SSU, maize Rubisco small subunit promoter; VTS, vacuolar targeting signal from maize aleurain. Only those events that had single copy transgene insertions are shown. The inset shows a Western blot obtained with the anti-Lox6 antibody on some of the events identified with asterisks. Western results confirm the ELISA results. The average expression in a non-transgenic line was 25 on the scale used on the Y axis.

FIG. 15: Remobilization of different proteins from the leaves of the To transgenic plants obtained with PEPC1-LOX6 gene construct. Abbreviations: Rubisco, Ribulose bisphosphate carboxylase; NR, nitrate reductase; PEP-C, phosphoenolpyruvate carboxylase.

FIG. 16: Expression of ZmLox6 in the field-grown T1 events derived from PEPC1PRO-Lox6 construct (FIG. 2A). Contains remobilization of different proteins after flowering in maize in transgenic events expressing ZmLox6 driven by the PEPC1 promoter. For each group, E indicates data associated with the control inbred line used for transformation. Each bar represents data from 128 plants across multiple events. Also shown are the expression levels of PEPC, Rubisco, and nitrate reductase proteins as quantitated by ELISA. The suffix E stands for the results from the inbred line used for transformation, which acts as a control. The ear leaf from each of the 16 field grown plants was sampled at weekly intervals across 8 events starting two week before flowering and ending four weeks later when the leaves had senesced. After extraction, proteins from the leaf samples were subjected to ELISA using antibodies against ZmLox6, ZmPEPC, Chlamydomonas Rubisco that we had shown specifically recognized both the maize Rubisco proteins, and maize nitrate reductase. The ELISA results are expressed on a relative scale with respect to the maximal value across transgenic or control plants being 100. The results clearly demonstrate a 5-fold higher level of expression of only the Lox6 protein in the transgenic events.

### DETAILED DESCRIPTION OF THE INVENTION

The present invention provides methods and compositions for increasing nitrogen storage capacity of a plant, thereby increasing nitrogen content in a plant or plant part thereof, compared to that obtainable with a wild-type or control plant. Methods of the invention comprise genetically altering a plant to express or overexpress a monocot-derived vegetative storage protein (VSP) or a biologically active fragment or variant thereof. Increasing expression of the monocot-derived VSP or fragment or variant thereof within the cells of a plant, particularly the vegetative cells, results in a plant with improved responsiveness to applied soil nitrogen and improved utilization of available soil nitrogen. Agronomic crop plants genetically modified in accordance with the methods disclosed herein beneficially mitigate problems associated with leaching and denitrification of nitrogen supplied to the soil in the form of fertilizers. By increasing nitrogen storage capacity within the cells of a plant, the methods of the invention provide for plants with increased nitrogen content, particularly within the leaves, stems, and seeds. The methods of the invention can thus be used to produce forage and silage crop plants with increased nutritional value, and to produce seed, particularly grain, with increased nutritional value.

According to the present invention, a VSP or a biologically active fragment or variant thereof is a polypeptide that has VSP properties, i.e., a polypeptide that serves as a reservoir to store excess nitrogen that may later be released and remobilized within the plant to support metabolism of existing plant tissues, for example, during periods of transient stress such as nutrient and/or water deficits, and/or to support growth and development of new tissues. A polypeptide that has VSP properties is referred to as a “VSP,” a “VSP polypeptide,” or a “VSP protein,” and a polynucleotide that encodes a polypeptide that has VSP properties is referred to as a “VSP polynucleotide.” By “monocot-derived” VSP or VSP polynucleotide is intended the VSP or VSP polynucleotide naturally occurs within a monocot species, or has been derived from a VSP or VSP polynucleotide that naturally occurs within a monocot species, where derivation is through genetic manipulation of the monocot VSP or VSP polynucleotide and/or the use of the monocot VSP polynucleotide to isolate VSP polynucleotides encoding homologous VSPs from other plant species.

In particular, monocot-derived VSP polynucleotides for use in the methods of the present invention include, for example, the coding sequence of the maize VSP-type lipoxygenase ZmLox6 gene as set forth in nucleotides 62-2737 of SEQ ID NO: 1 or in SEQ ID NO: 3, sequences encoding the ZmLox6 protein set forth in SEQ ID NO: 2, and fragments and variants thereof as defined below. Monocot-derived VSP polypeptides of the present invention include, for example, the ZmLox6 protein set forth in SEQ ID NO: 2 and biologically active fragments and variants thereof as defined herein below.

As described more fully in the Experimental section elsewhere herein, the ZmLox6 protein exhibits the characteristics of a VSP, and thus represents a VSP-type lipoxygenase. For example, the ZmLox6 protein is induced upon supplying high levels of N in the growth medium and is most highly expressed in the leaves, in a manner similar to the soybean VSP referred to as VLX-D (Tranbarger, et al., (1991) Plant Cell 3:973-988). In view of its VSP properties, ZmLox6 is referred to herein as a VSP, and sequences encoding ZmLox6 are considered to be VSP polynucleotides. Although the ZmLox6 protein exhibits VSP properties and is thus a VSP-type lipoxygenase, it is recognized that the ZmLox6 protein or variants thereof may also exhibit other biological activities associated with other members of the lipoxygenase family of proteins (see, for example, U.S. Pat. No. 6,921,847; herein incorporated by reference in its entirety).

According to the present invention, “increasing nitrogen storage capacity” of a plant, or plant part or plant cell thereof, refers to an increase in the total soluble protein fraction of the plant, or plant part or plant cell thereof, of at least 1%, 5%, 10%, 20%, 30%, 40% or 50% relative to that observed with a wild-type or control plant, or plant part or plant cell thereof, respectively. By increasing nitrogen storage capacity, particularly within the leaf and stem tissues, the nitrogen content, and thus nutritional value, of a forage or silage crop plant can be increased.

Forage is herbaceous plant material (including grasses and legumes) eaten by grazing animals, while silage is fermented, high-moisture forage typically fed to ruminant animals. Plants used in silage production include corn, grain sorghum (Milo), perennial grasses (such as Bermudagrass, Stargrass, and Limpograss (Hemarthria)), annual grasses (such as forage sorghum, sorghum-sudan hybrids, pearlmillet, and small grains and ryegrass), legumes (such as alfalfa, red clover and other cool season legumes, and summer legumes including hairy indigo, alyce clover, aeschynomene, and rhizome perennial peanut), sugarcane, oats, and crop combinations such as grain sorghum and soybeans or oats and peas.

Although corn is a primary source of silage for cattle and dairy feed, corn silage is relatively low in protein content and must be supplemented with higher protein content feed such as from soybean meal. Although soybeans produce vegetative plant tissue with much higher nitrogen levels than found in corn, soybean is not suitable for silage production. Therefore, developing monocots with increased expression of VSP polypeptides such as ZmLox6 or biologically active variants thereof, would improve nitrogen-sequestration and nutritional value of forage and silage crops.

According to the present invention, “increasing nitrogen content of a plant, or plant part thereof,” used for forage and silage refers to an increase in the % total nitrogen within the plant or plant part thereof as measured on a dry weight basis of at least 1%, 2%, 5%, 10%, 20% or 50% relative to that observed for a wild-type or control plant, or plant part thereof. Where the seed is of agronomic interest, such as in grain crops, the methods of the invention can increase seed yield, and/or increase seed nitrogen content, and/or increase seed nutritional value relative to seed obtained from a native control plant, as excess nitrogen sequestered within leaf and stem tissues in the form of the ZmLox6 protein or variant thereof can be remobilized to support greater seed production and seed fill, particularly when soil nitrogen levels are limiting to reproductive sink development. According to the present invention, “increasing nitrogen content of seed” refers to an increase in the % nitrogen within seed as measured on a seed dry weight basis of at least 1%, 2%, 5%, 10%, 20%, or 50% relative to that observed for seed of a wild-type or control plant.

The methods of the present invention comprise increasing the expression of monocot-derived VSPs in plants, particularly expression of the maize VSP-type lipoxygenase ZmLox6 or biologically active fragment or variant thereof having VSP properties. Thus, in some embodiments, the methods comprise introducing into a plant of interest at least one nucleotide construct comprising a nucleotide sequence encoding the ZmLox6 protein or a biologically active fragment or variant thereof operably linked to a promoter that drives expression in a plant cell. The nucleotide construct may optionally comprise an operably linked coding sequence for a vacuolar sorting signal or plastid transit peptide in order to direct the ZmLox6 protein or fragment or variant thereof into a vacuolar compartment or plastid compartment, respectively, of the plant cells in which this protein is expressed. In particular embodiments, the VSP is ZmLox6 or biologically active fragment or variant thereof and the plant is a monocot such as maize.

Any promoter can be used to drive expression of the monocot-derived VSP, for example, the ZmLox6 protein or biologically active fragment or variant thereof having VSP properties, including, but not limited to, the promoters described herein below. Thus, for example, in some embodiments, expression of the VSP, for example, the ZmLox6 protein or biologically active fragment or variant thereof, is driven by a constitutive promoter to provide for expression in the cells throughout a plant at most times and in most tissues, or an inducible promoter so that expression is induced in response to a stimulus, for example in response to wounding, externally applied chemicals, or environmental stress. In other embodiments, expression of the VSP, for example, the ZmLox6 protein or biologically active fragment or variant thereof, is driven by a tissue-preferred promoter such that expression occurs preferentially within a desired tissue. In one such embodiment, the promoter is a leaf-preferred promoter to provide for preferential expression within the cells of the leaf tissues.

In yet other embodiments, the promoter is chosen to provide for expression of the VSP, for example, ZmLox6 protein or biologically active fragment or variant thereof, preferentially within specific leaf cells, for example, in the mesophyll cells or bundle-sheath cells, to provide for localized accumulation of the VSP or fragment or variant thereof within these cells of the leaf tissue. Such promoters are referred to herein as “mesophyll cell-preferred promoters” or “bundle-sheath cell-preferred promoters,” and include those promoters described elsewhere herein. Though leaf tissues of C3 plants generally comprise loosely organized bundle-sheath cells, the bulk of the photosynthetic enzymes and associated photosynthetic machinery is contained within the chloroplasts of the more abundant mesophyll cells. Where preferential expression of the VSP or biologically active fragment or variant thereof is targeted within the mesophyll cells of the leaves of a C3 plant, the nucleotide construct comprising the coding sequence for the VSP of interest or fragment or variant thereof operably linked to a mesophyll cell-preferred promoter can optionally comprise a vacuolar sorting signal to direct the expressed VSP or fragment or variant thereof into the vacuolar compartment of these cells to minimize impact on chloroplast and cellular function.

The distinct division of photosynthetic functions between mesophyll and bundle-sheath cells of C4 plants presents different nitrogen reservoir opportunities that can advantageously be manipulated to increase nitrogen storage capacity of these plants. The less abundant chloroplasts within mesophyll cells of a C4 plant such as maize contain little or no Rubisco, which is concentrated within the abundant chloroplasts of the bundle-sheath cells. Without being bound by theory, the plastidial compartment of mesophyll cells within the leaves of a C4 plant can be expected to provide an extra reservoir for storage of nitrogen in the form of a monocot-derived VSP or fragment or variant thereof beyond that provided by the cytoplasmic and vacuolar compartments found in both the mesophyll and bundle-sheath cells of C4 plant leaf tissues, while minimally impacting chloroplast function.

It is recognized that preferential expression within both the mesophyll and bundle-sheath cells of a C4 plant may be desirable. This can be accomplished, for example, by introducing into the plant, either as a single nucleotide construct or as multiple nucleotide constructs, at least one polynucleotide that comprises the coding sequence of the VSP of interest or fragment or variant thereof operably linked to a promoter that preferentially drives expression of the VSP or fragment or variant thereof within the mesophyll cells, and at least another polynucleotide that comprises a coding sequence for the VSP of interest or fragment or variant thereof operably linked to a promoter that drives expression of the VSP or fragment or variant thereof within the bundle-sheath cells. Where the VSP or fragment or variant thereof is to be expressed preferentially within the mesophyll and/or bundle-sheath cells of the C4 plant, for example, maize, the nucleotide construct(s) can optionally comprise an operably linked coding sequence for a vacuolar sorting signal to direct the expressed VSP or fragment or variant thereof into the vacuolar compartment of the mesophyll or bundle-sheath cell. Where the VSP or fragment or variant thereof is to be preferentially expressed within the mesophyll cells of a C4 plant, alone or in combination with preferential expression in the bundle-sheath cells, the nucleotide construct to be introduced into the plant can be designed such that the polynucleotide encodes an operably linked vacuolar transit peptide as noted above, or can be designed such that the polynucleotide encodes an operably linked plastid transit peptide, for example, a chloroplast transit peptide, to direct the expressed VSP or fragment or variant thereof into the plastid compartment of the mesophyll cells.

By increasing expression of a monocot-derived VSP, for example, the ZmLox6 protein or biologically active fragment or variant thereof, within a plant, nitrogen storage capacity within the plant can be increased, yielding an overall increase in total plant nitrogen content within one or more tissues of interest. In this manner, the methods of the invention find use in increasing total nitrogen content and nutritional value of plants that are utilized for forage and silage, and increasing total nitrogen content and nutritional value of seed, for example, in grain crops.

Though the coding sequences for the monocot-derived VSP described herein and biologically active fragments and variants thereof can be used to increase nitrogen storage capacity of any plant of interest, the ZmLox6 coding sequence, and fragments and variants thereof, find particular use in increasing nitrogen storage capacity, tissue nitrogen content, and nutritional value of a monocot plant, for example maize, as this VSP has evolved to function within the monocot cellular environment. It is further recognized that increasing the nitrogen storage capacity of a plant can beneficially provide for more efficient nitrogen utilization from the environment while providing the plant with excess nitrogen reserves that can be mobilized during later periods of plant development, such as during seed set and seed fill, particularly when the plant is subjected to water and/or nutrient stress.

The methods of the invention encompass the use of isolated or substantially purified VSP polynucleotide or protein compositions, including the ZmLox6 coding sequence and protein, in order to increase nitrogen storage capacity of a plant, to increase nitrogen content and nutritional value of a forage or silage crop plant, and to increase nitrogen content and nutritional value of seed, particularly grain of agronomic crop plants. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5% or 1% (by dry weight) of contaminating protein. When the protein of the invention or biologically active portion thereof is recombinantly produced, optimally culture medium represents less than about 30%, 20%, 10%, 5% or 1% (by dry weight) of chemical precursors or non-protein-of-interest chemicals.

The use of fragments and variants of monocot-derived VSP polynucleotides and polypeptides encoded thereby is also encompassed by the present invention. Depending on the context, “fragment” refers to a portion of the polynucleotide or a portion of the amino acid sequence and hence protein encoded thereby. Fragments of a polynucleotide may encode protein fragments that retain the biological activity of the original protein and hence confer VSP properties. Thus, fragments of a nucleotide sequence may range from at least about 20 nucleotides, about 50 nucleotides, about 100 nucleotides and up to the full-length polynucleotide encoding a VSP polypeptide.

A fragment of a VSP polynucleotide that encodes a biologically active portion of a VSP polypeptide will encode at least 15, 25, 30, 50, 100, 150, 200, 250, 300, 350, 400, 450, 500, 550, 600, 650, 700, 750, 800, 850 or 875 contiguous amino acids, or up to the total number of amino acids present in a full-length VSP polypeptide (for example, 892 amino acids for the ZmLox6 polypeptide of SEQ ID NO: 2). A portion of a VSP polypeptide that may carry the characteristics of the whole protein can be prepared by isolating a portion of a VSP polynucleotide, expressing the encoded portion of the VSP polypeptide (e.g., by recombinant expression in vitro), and assessing the activity of the encoded portion of the VSP polypeptide. Polynucleotides that are fragments of a VSP polynucleotide comprise at least 16, 20, 50, 75, 100, 150, 200, 250, 300, 350, 400, 450, 500, 550, 600, 650, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600 or 2,650 contiguous nucleotides, or up to the number of nucleotides present in a full-length VSP polynucleotide (for example, 2,909 contiguous nucleotides for the ZmLox6 nucleotide sequence of SEQ ID NO: 1 or 2,676 contiguous nucleotides for the ZmLox6 coding sequence of SEQ ID NO: 3).

The term “variants” refers to substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of a VSP polypeptide, for example, ZmLox6 of SEQ ID NO: 2. Naturally occurring allelic variants such as these can be identified with the use of well-known molecular biology techniques, as, for example, with polymerase chain reaction (PCR) and hybridization techniques. Variant polynucleotides also include synthetically derived polynucleotides, such as those generated, for example, by using site-directed mutagenesis or “shuffling.” Generally, variants of a particular polynucleotide, for example, the ZmLox6 sequence set forth in SEQ ID NO: 1, or the ZmLox6 coding sequence set forth in nucleotides 62-2737 of SEQ ID NO: 1 or in SEQ ID NO: 3, have at least about 40%, 45%, 50%, 55%, 60%, 65%, 70%, 75%, 80%, 85%, 90%, 91 %, 92%, 93%, 94%, 95%, 96%, 97%, 98%, 99% or more sequence identity to that particular polynucleotide as determined by sequence alignment programs and parameters as described elsewhere herein.

Variants of a particular polynucleotide (i.e., the reference polynucleotide) can also be evaluated by comparison of the percent sequence identity between the polypeptide encoded by a variant polynucleotide and the polypeptide encoded by the reference polynucleotide. Thus, for example, in one embodiment, the variant of a VSP polynucleotide is an isolated polynucleotide that encodes a VSP polypeptide having a given percent identity to the ZmLox6 polypeptide of SEQ ID NO: 2. Percent sequence identity between any two polypeptides can be calculated using sequence alignment programs and parameters described elsewhere herein. Where any given pair of polynucleotides used to practice the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from a native and/or original protein by deletion (so-called truncation) of one or more amino acids at the N-terminal and/or C-terminal end of the protein; deletion and/or addition of one or more amino acids at one or more internal sites in the protein; or substitution of one or more amino acids at one or more sites in the protein. Variant proteins encompassed by the present invention are biologically active, that is they continue to possess the desired VSP properties as described herein. Biologically active variants of a VSP polypeptide, for example, the ZmLox6 protein shown in SEQ ID NO: 2, will have at least about 40%, 45%, 50%, 55%, 60%, 65%, 70%, 75%, 80%, 85%, 90%, 91 %, 92%, 93%, 94%, 95%, 96%, 97%, 98%, 99% or more sequence identity to the amino acid sequence for the native protein as determined by sequence alignment programs and parameters described elsewhere herein. A biologically active variant of a VSP polypeptide, for example, the ZMLox6 protein, may differ from that polypeptide by as few as 1-15 amino acid residues, as few as 1 -10, such as 6-10, as few as 5, as few as 4, 3, 2, or even 1 amino acid residue.

The monocot-derived VSP polypeptides for use in practicing the invention may be altered in various ways including amino acid substitutions, deletions, truncations, and insertions. Methods for such manipulations are generally known in the art. For example, amino acid sequence variants and fragments of the ZmLox6 protein of SEQ ID NO: 2 can be prepared by mutations in the encoding polynucleotide, for example, the sequence set forth in SEQ ID NO: 1, or the coding sequence set forth in nucleotides 62-2737 of SEQ ID NO: 1 or in SEQ ID NO: 3. Methods for mutagenesis and polynucleotide alterations are well known in the art. See, for example, Kunkel (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to amino acid substitutions that do not affect biological activity of the protein of interest may be found in the model of Dayhoff, et al., (1978) Atlas of Protein Sequence and Structure (Natl. Biomed. Res. Found., Washington, D.C.), herein incorporated by reference. Conservative substitutions, such as exchanging one amino acid with another having similar properties, may be made.

The monocot-derived VSP polypeptides, for example, the ZMLox6 protein, or biologically active fragments and variants thereof, may also be altered by modifying the encoding polynucleotide to express a VSP polypeptide enriched in essential amino acids, including lysine, methionine, tryptophan, threonine, phenylalanine, leucine, valine, and isoleucine relative to average levels of such amino acids in the native protein. In one embodiment, a polynucleotide encoding the ZMLox6 protein, or biologically active fragment or variant thereof, is modified such that the protein is enriched for lysine content. Methods for altering nutritional amino acid content of a protein are known (see, e.g., U.S. Pat. No. 6,905,877, herein incorporated by reference in its entirety). Such methods therefore find use in improving the nutritional value of VSP polypeptides described herein, as well as improving the nutritional value of plants, or plant parts thereof, expressing such nutritionally enhanced VSP polypeptides.

Variant VSP polynucleotides and VSPs for use in the methods of the invention also encompass sequences and proteins derived from a mutagenic and recombinogenic procedure such as DNA shuffling. With such a procedure, one or more different VSP polypeptide coding sequences can be manipulated to create a new VSP polypeptide possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the ZMLox6 sequence of SEQ ID NO: 1 or SEQ ID NO: 3 and other known Lox genes to obtain a new gene coding for a VSP protein with an improved property of interest, such as increased content of essential amino acids. Strategies for such DNA shuffling are known in the art. See, for example, Stemmer (1994) Proc. Natl. Acad. Sci. USA 91:10747-10751; Stemmer (1994) Nature 370:389-391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291; and U.S. Pat. Nos. 5,605,793 and 5,837,458.

The ZmLox6 polynucleotide for use in the methods of the invention can be used to isolate corresponding VSP sequences from other plants, including other monocots. In this manner, methods such as PCR, hybridization, and the like can be used to identify such sequences based on their sequence homology to the ZmLox6 sequence set forth in SEQ ID NO: 1, or the ZmLox6 coding sequence set forth in nucleotides 62-2470 of SEQ ID NO: 1 or in SEQ ID NO: 3. Sequences isolated based on their sequence identity to the entire ZmLox6 nucleotide sequence set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 94%, 95%, 96%, 97%, 98%, 99% or greater sequence identity. Functions of orthologs are often highly conserved among species. Thus, isolated polynucleotides that encode for a VSP polypeptide and which hybridize under stringent conditions to the ZmLox6 sequence of SEQ ID NO: 1, or the ZmLox6 coding sequence set forth in nucleotides 62-2737 of SEQ ID NO: 1 or in SEQ ID NO: 3, or to variants or fragments thereof, can be used to practice the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook, et al., (1989) Molecular Cloning: A Laboratory Manual (2d ed., Cold Spring Harbor Laboratory Press, Plainview, New York). See also, Innis, et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially mismatched primers, and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the ZmLox6 nucleotide sequence of SEQ ID NO: 1, or the ZmLox6 coding sequence set forth in nucleotides 62-2737 of SEQ ID NO: 1 or in SEQ ID NO: 3. Methods for preparation of probes for hybridization and for construction of cDNA and genomic libraries are generally known in the art and are disclosed in Sambrook, et al., (1989) Molecular Cloning: A Laboratory Manual (2d ed., Cold Spring Harbor Laboratory Press, Plainview, New York).

For example, the entire ZmLox6 polynucleotide disclosed in SEQ ID NO: 1, nucleotides 62-2737 of SEQ ID NO: 1, or SEQ ID NO: 3, or one or more portions thereof, may be used as a probe capable of specifically hybridizing to corresponding VSP polynucleotides and messenger RNAs. To achieve specific hybridization under a variety of conditions, such probes include sequences that are unique among VSP polynucleotide sequences and are optimally at least about 10 nucleotides in length, and most optimally at least about 20 nucleotides in length. Such probes may be used to amplify corresponding VSP polynucleotides from a chosen plant by PCR. This technique may be used to isolate additional VSP coding sequences from a desired plant or as a diagnostic assay to determine the presence of VSP coding sequences in a plant. Hybridization techniques include hybridization screening of plated DNA libraries (either plaques or colonies; see, for example, Sambrook, et al., (1989) Molecular Cloning: A Laboratory Manual (2d ed., Cold Spring Harbor Laboratory Press, Plainview, New York).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1 % SDS at 37° C., and a wash in 0.5× to 1×SSC at 55 to 60° C. Exemplary high stringency conditions include hybridization in 50% formamide, 1 M NaCl ,1 % SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel, et al., eds. (1995) Current Protocols in Molecular Biology, Chapter 2 (Greene Publishing and Wiley-Interscience, New York). See, Sambrook, et al., (1989) Molecular Cloning: A Laboratory Manual (2d ed., Cold Spring Harbor Laboratory Press, Plainview, New York).

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50,100 or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith, et al., (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 87:2264-2268, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244 (1988); Higgins, et al., (1989) CABIOS 5:151-153; Corpet, et al., (1988) Nucleic Acids Res. 16:10881-90; Huang, et al., (1992) CABIOS 8:155-65; and Pearson, et al., (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul, et al., (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a VSP for use in the methods of the present invention. BLAST protein searches can be performed with the BLASTX program, score=50, wordlength=3, to obtain amino acid sequences homologous to a VSP for use in the methods of the present invention. To obtain gapped alignments for comparison purposes, Gapped BLAST (in BLAST 2.0) can be utilized as described in Altschul, et al., (1997) Nucleic Acids Res. 25:3389. Alternatively, PSI-BLAST (in BLAST 2.0) can be used to perform an iterated search that detects distant relationships between molecules. See, Altschul, et al., (1997) supra. When utilizing BLAST, Gapped BLAST, PSI-BLAST, the default parameters of the respective programs (e.g., BLASTN for nucleotide sequences, BLASTX for proteins) can be used. BLAST software is publicly available on the NCBI website. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see, Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The use of the term “polynucleotide” is not intended to be limited to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. Thus, polynucleotides also encompass all forms of sequences including, but not limited to, single-stranded forms, double-stranded forms, hairpins, stem-and-loop structures, and the like.

The VSP polynucleotide, for example, the ZmLox6 polynucleotide or fragment or variant thereof, can be provided in expression cassettes for expression in the plant of interest. The cassette will include 5′ and 3′ regulatory sequences operably linked to the VSP polynucleotide. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by “operably linked” is intended that the coding regions are in the same reading frame. The cassette may additionally contain at least one additional gene to be cotransformed into the plant. Alternatively, the additional gene(s) can be provided on multiple expression cassettes. Such an expression cassette is provided with a plurality of restriction sites and/or recombination sites for insertion of the VSP polynucleotide to be under the transcriptional regulation of the regulatory regions. The expression cassette may additionally contain other genes, including other selectable marker genes.

The expression cassette will include in the 5′-3′ direction of transcription a transcriptional and translational initiation region (i.e., a promoter), the VSP polynucleotide, for example, SEQ ID NO: 1, nucleotides 62-2737 of SEQ ID NO: 1, SEQ ID NO: 3, or fragment or variant thereof, and a transcriptional and translational termination region (i.e., termination region) functional in plants. The regulatory regions (i.e., promoters, transcriptional regulatory regions, and translational termination regions) and/or the VSP polynucleotide may be native/analogous to the host cell or to each other. Alternatively, the regulatory regions and/or the VSP polynucleotide may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

While it may be optimal to express the VSP polynucleotides using heterologous promoters, the native promoter sequences may be used. Such constructs can change expression levels of the encoded polypeptide in the plant or plant cell. Thus, the phenotype of the plant or cell can be altered.

The termination region may be native with the transcriptional initiation region, may be native with the operably linked VSP polynucleotide of interest, may be native with the plant host, or may be derived from another source (i.e., foreign or heterologous) to the promoter, the VSP polynucleotide of interest, the plant host, or any combination thereof. Convenient termination regions for use in the present invention include those available from the Ti-plasmid of A. tumefaciens, such as the octopine synthase and nopaline synthase termination regions. See also, Guerineau, et al., (1991) Mol. Gen. Genet. 262:141-144; Proudfoot (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903; and Joshi, et al., (1987) Nucleic Acids Res. 15:9627-9639.

In some embodiments of the invention, the expression cassette comprises a coding sequence for a vacuolar sorting signal operably linked to the coding sequence for the VSP of interest, for example, ZmLox6 of SEQ ID NO: 2 or biologically active fragment or variant thereof. Of particular interest are sorting signals that sort proteins to protein storage vacuoles. See, for example, Neuhaus and Rogers (1998) Plant Mol. Biol. 38:127-144, and Holwerda, et al., (1992) The Plant Cell 4:307-318, herein incorporated by reference. Examples of such coding sequences for vacuolar sorting signals are known in the art and include, but are not limited to, the maize proaleurain vacuolar sorting signal. For example, C-terminal propeptides from tobacco chitinase and pumpkin 2S albumin have both been successfully used to target soluble proteins to the vacuole. See, Mistubishi, et al., (2000) Plant Cell Physiol. 41(9):993-1001; and Tamura, et al., (2003) The Plant J. 35:545-555.

In other embodiments, the expression cassette comprises a coding sequence for a plastid transit peptide operably linked to the coding sequence for the VSP of interest, for example, ZmLox6 of SEQ ID NO: 2 or biologically active fragment or variant thereof, in order to direct the expressed VSP into the plastid compartment of the plant cells in which the VSP is expressed. Such transit peptides are known in the art. See, for example, Von Heijne, et al., (1991) Plant Mol. Biol. Rep. 9:104-126; Clark, et al., (1989) J. Biol. Chem. 264:17544-17550; Della-Cioppa, et al., (1987) Plant Physiol. 84:965-968; Romer, et al., (1993) Biochem. Biophys. Res. Commun. 196:1414-1421; and Shah, et al., (1986) Science 233:478-481. Chloroplast transit peptides (also referred to as chloroplast targeting sequences) are known in the art and include the chloroplast small subunit of ribulose-1,5-bisphosphate carboxylase (Rubisco) (de Castro Silva Filho, et al., (1996) Plant Mol. Biol. 30:769-780; Schnell, et al., (1991) J. Biol. Chem. 266(5):3335-3342); 5-(enolpyruvyl)shikimate-3-phosphate synthase (EPSPS) (Archer, et al., (1990) J. Bioenerg. Biomemb. 22(6):789-810); tryptophan synthase (Zhao, et al., (1995) J. Biol. Chem. 270(11):6081 -6087); plastocyanin (Lawrence, et al., (1997) J. Biol. Chem. 272(33):20357-20363); chorismate synthase (Schmidt, et al., (1993) J. Biol. Chem. 268(36):27447-27457); and the light harvesting chlorophyll a/b binding protein (LHBP) (Lamppa, et al., (1988) J. Biol. Chem. 263:14996-14999). See also Von Heijne, et al., (1991) Plant Mol. Biol. Rep. 9:104-126; Clark, et al., (1989) J. Biol. Chem. 264:17544-17550; Della-Cioppa, et al., (1987) Plant Physiol. 84:965-968; Romer, et al., (1993) Biochem. Biophys. Res. Commun. 196:1414-1421; and Shah, et al., (1986) Science 233:478-481.

Methods are known in the art for increasing expression of a polypeptide of interest in a plant or plant cell, for example, by inserting into the polypeptide coding sequence one or two G/C-rich codons (such as GCG or GCT) immediately adjacent to and downstream of the initiating methionine ATG codon. Where appropriate, the VSP polynucleotides may be optimized for increased expression in the transformed plant. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray, et al., (1989) Nucleic Acids Res. 17:477-498, herein incorporated by reference. Embodiments comprising such modifications are also a feature of the invention.

Additional sequence modifications are known to enhance gene expression in a particular plant host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given plant host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein, et al., (1989) Proc. Natl. Acad. Sci. USA 86:6126-6130); potyvirus leaders, for example, TEV leader (Tobacco Etch Virus) (Gallie, et al., (1995) Gene 165(2):233-238), MDMV leader (Maize Dwarf Mosaic Virus) (Virology 154:9-20), and human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) in Molecular Biology of RNA, ed. Cech (Liss, New York), pp. 237-256); and maize chlorotic mottle virus leader (MCMV) (Lommel, et al., (1991) Virology 81:382-385). See also, Della-Cioppa, et al., (1987) Plant Physiol. 84:965-968.

In preparing the expression cassette, the various polynucleotide fragments may be manipulated, so as to provide for sequences to be in the proper orientation and, as appropriate, in the proper reading frame. Toward this end, adapters or linkers may be employed to join the fragments or other manipulations may be involved to provide for convenient restriction sites, removal of superfluous material such as the removal of restriction sites, or the like. For this purpose, in vitro mutagenesis, primer repair, restriction, annealing, resubstitutions, e.g., transitions and transversions, may be involved. Standard recombinant DNA and molecular cloning techniques used herein are well known in the art and are described more fully, for example, in Sambrook, et al., Molecular Cloning: A Laboratory Manual (Cold Spring Harbor Laboratory Press; Plainview, N.Y.).

A number of promoters can be used in the practice of the invention, including the native promoter of the VSP polynucleotide sequence of interest. The promoters can be selected based on the desired outcome. The VSP polynucleotides of interest can be combined with constitutive, inducible, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); PEMU (Last, et al., (1991) Theor. Appl. Genet. 81:581-588); MAS (Velten, et al., (1984) EMBO J. 3:2723-2730); ALS promoter (U.S. Pat. No. 5,659,026), and the like. Other constitutive promoters include, for example, those described in U.S. Pat. Nos. 5,608,149; 5,608,144; 5,604,121; 5,569,597; 5,466,785; 5,399,680; 5,268,463; 5,608,142; and 6,177,611.

Additionally, a wound-inducible promoter may be used in the constructions of the invention. Such wound-inducible promoters include promoters for the potato proteinase inhibitor (pin II) gene (Ryan (1990) Ann. Rev. Phytopath. 28:425-449; Duan, et al., (1996) Nature Biotechnology 14:494-498); wun1 and wun2, U.S. Pat. No. 5,428,148; win1 and win2 (Stanford, et al., (1989) Mol. Gen. Genet. 215:200-208); systemin (McGurl, et al., (1992) Science 225:1570-1573); WIP1 (Rohmeier, et al., (1993) Plant Mol. Biol. 22:783-792; Eckelkamp, et al., (1993) FEBS Letters 323:73-76); MPI gene (Corderok, et al., (1994) Plant J. 6(2):141-150); and the like, herein incorporated by reference.

Tissue-preferred promoters can be utilized to target enhanced VSP polypeptide expression within a particular plant tissue. Tissue-preferred promoters include those disclosed in Yamamoto, et al., (1997) Plant J. 12(2):255-265; Kawamata, et al., (1997) Plant Cell Physiol. 38(7):792-803; Hansen, et al., (1997) Mol. Gen Genet. 254(3):337-343; Russell, et al., (1997) Transgenic Res. 6(2):157-168; Rinehart, et al., (1996) Plant Physiol. 112(3):1331-1341; Van Camp, et al., (1996) Plant Physiol. 112(2):525-535; Canevascini, et al., (1996) Plant Physiol. 112(2):513-524; Yamamoto, et al., (1994) Plant Cell Physiol. 35(5):773-778; Lam (1994) Results Probl. Cell Differ. 20:181-196; Orozco, et al., (1993) Plant Mol Biol. 23(6):1129-1138; Matsuoka, et al., (1993) Proc Natl. Acad. Sci. USA 90(20):9586-9590; and Guevara-Garcia, et al., (1993) Plant J. 4(3):495-505. Such promoters can be modified, if necessary, for weak expression.

Leaf-preferred promoters are known in the art. See, for example, Yamamoto, et al., (1997) Plant J. 12(2):255-265; Kwon, et al., (1994) Plant Physiol. 105:357-67; Yamamoto, et al., (1994) Plant Cell Physiol. 35(5):773-778; Gotor, et al., (1993) Plant J. 3:509-18; Orozco, et al., (1993) Plant Mol. Biol. 23(6):1129-1138; and Matsuoka, et al., (1993) Proc. Natl. Acad. Sci. USA 90(20):9586-9590.

In some embodiments, the VSP polypeptide, for example, ZmLox6 of SEQ ID NO: 2 or biologically active fragment or variant thereof, is expressed preferentially within specific leaf cells, particularly within the mesophyll cells, bundle-sheath cells, or both. Promoters that provide for mesophyll cell-preferred expression of operably linked heterologous polynucleotides in transgenic plants include, but are not limited to, promoters for phosphoenolpyruvate carboxylase (PEP carboxylase) and pyruvate; orthophosphate dikinase genes (see, for example, Matsuoka and Sanada (1991) Mol. Gen. Genet. 225(3):411-419; Matsuoka, et al., (1993) Proc. Natl. Acad. Sci. 90:9586-9590; Kausch, et al., (2001) Plant Mol. Biol. 45(1):1-15; Taniguchi, et al., (2000) Plant Cell Physiol. 41(1):42-48); promoters for cab-i genes (see, for example, the promoter for the maize cab-m1 gene, in Shiina, et al., (1997) Plant Physiol. 115(2):477-483 and Bansal, et al., (1992) Proc. Natl. Acad. Sci. 89:3654-3658); and promoters for Rubisco small subunit genes (see, for example, the promoters for the tomato and rice rbcS genes, in Kyozuka, et al., (1993) Plant Physiol. 102:991-1000; and mesophyll cell-preferred expression provided by the promoter for the maize Rubisco small subunit gene within a transgenic C3 plant (see, for example, Matsuoka and Sanada (1991) Mol. Gen. Genet. 225(3):411-419)). Promoters that provide for bundle-sheath cell-preferred expression of operably linked heterologous polynucleotides in transgenic plants include, but are not limited to, promoters for the Rubisco small unit genes of C4 plants (see, for example, the maize rbcS-m3 promoter and elements providing for bundle-sheath cell-specific expression, described in Viret, et al., (1994) Proc. Natl. Acad. Sci. USA 91:8577-8581, Bansal, et al., (1992) Proc. Natl. Acad. Sci. USA 89:3654-3658), and Schaffner and Sheen (1991) Plant Cell 3:997-1012.

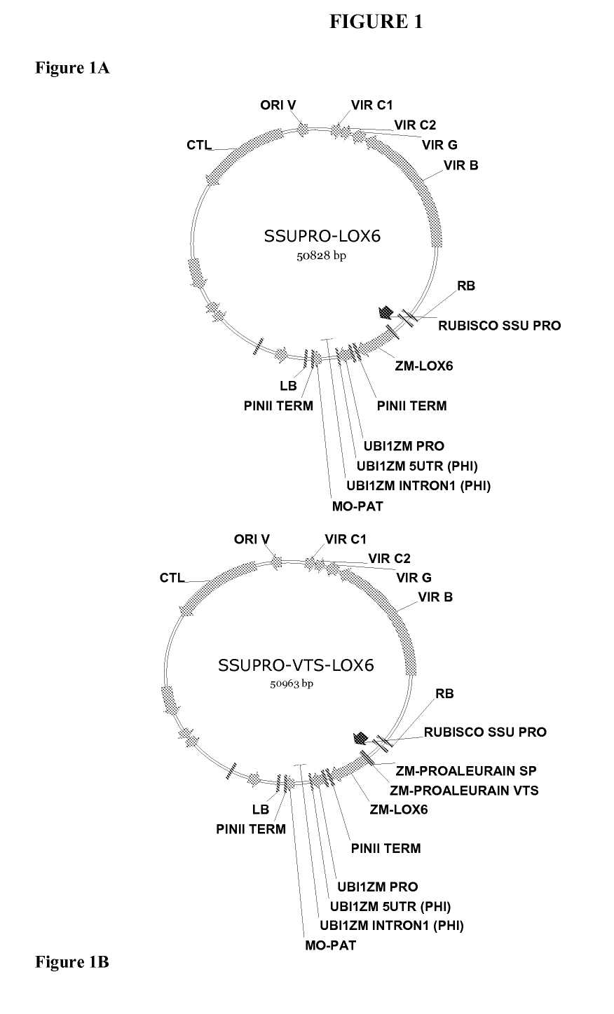

In some embodiments, the expression cassette is designed such that expression of the encoded VSP, for example ZmLox6 of SEQ ID NO: 2 or biologically active fragment or variant thereof, is driven by the maize Rubisco small subunit (SSU) promoter (see, for example, FIG. 1A; also see Genbank Accession number U09743.1). When introduced into a C4 plant such as maize, this construct provides for preferential expression of the encoded VSP within the bundle-sheath cells of the leaf tissues. In other embodiments, this construct further comprises a coding sequence for a vacuolar sorting signal, for example, the maize proaleurain vacuolar sorting signal, operably linked to the VSP polynucleotide so that the expressed VSP is directed to the vacuolar compartment of the bundle-sheath cell (see, for example, FIG. 1B). ZM-proaleurain signal peptide (SP) and vacuolar targeting sequence (VTS) are necessary for Golgi-mediated processing and vacuole targeting of ZmLox6.

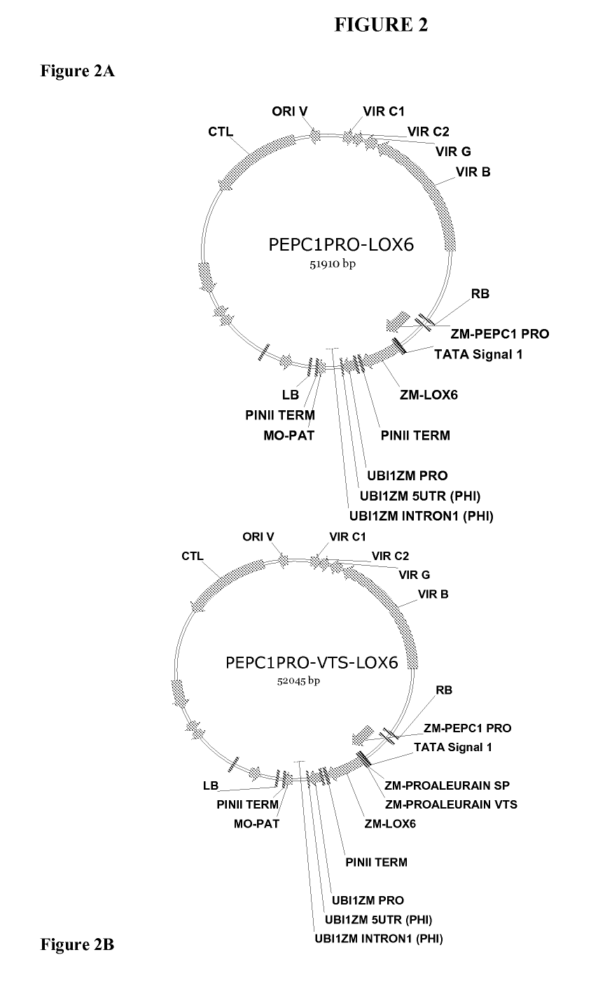

In some embodiments, the expression cassette is designed such that expression of the encoded VSP, for example ZmLox6 of SEQ ID NO: 2 or biologically active fragment or variant thereof, is driven by the maize phosphoenolpyruvate carboxylase (PEPC1) promoter (see, for example, FIG. 2A; also see GenBank Accession number X15642.1 (partial sequence)). When introduced into a plant, this construct provides for preferential expression of the encoded VSP within the mesophyll cells of the leaf tissue. In other embodiments, this construct further comprises a coding sequence for a vacuolar sorting signal, for example, the maize proaleurain vacuolar sorting signal, operably linked to the VSP polynucleotide so that the expressed VSP is directed into the vacuolar compartment of the mesophyll cell (see, for example, FIG. 2B). Where the plant is a C4 plant such as maize, the expression cassette can alternatively comprise a coding sequence for a plastid transit peptide, for example, a chloroplast transit peptide, operably linked to the VSP polynucleotide so that the expressed VSP is directed into the plastid compartment of the mesophyll cell.

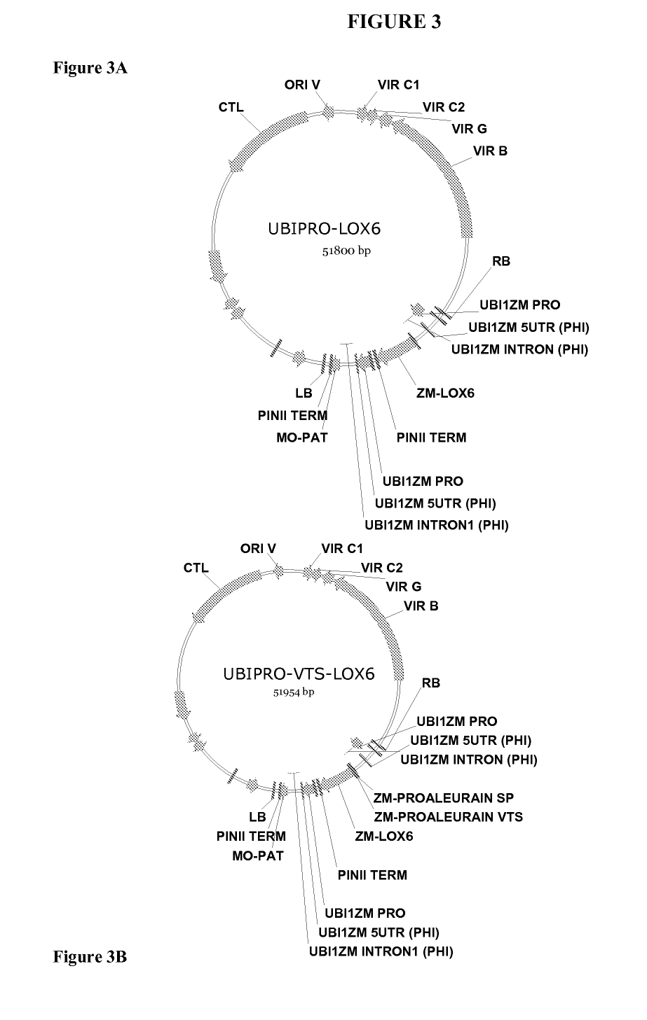

In some embodiments, the expression cassette is designed such that expression of the encoded VSP, for example ZmLox6 of SEQ ID NO: 2 or biologically active fragment or variant thereof, is driven by a constitutive promoter such as a ubiquitin (UBI) promoter, for example, the maize UBI promoter (see, for example, FIG. 3A; also see Genbank Accession number S94464). In other embodiments, the expression cassette also comprises a coding sequence for a vacuolar sorting signal, for example, the maize proaleurain vacuolar sorting signal, operably linked to the VSP polynucleotide so that the expressed VSP is directed into the vacuolar compartment of the cell (see, for example, FIG. 3B).

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su, et al., (2004) Biotechnol. Bioeng. 85:610-9 and Fetter, et al., (2004) Plant Cell 16:215-28), cyanofluorescent protein (CYP) (Bolte, et al., (2004) J. Cell Science 117:943-54 and Kato, et al., (2002) Plant Physiol 129:913-42), and yellow fluorescent protein (PhiYFP™ from Evrogen, see, Bolte, et al., (2004) J. Cell Science 117:943-54). For additional selectable markers, see generally, Yarranton (1992) Curr. Opin. Biotech. 3:506-511; Christopherson, et al., (1992) Proc. Natl. Acad. Sci. USA 89:6314-6318; Yao, et al., (1992) Cell 71:63-72; Reznikoff (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Sci. USA 86:5400-5404; Fuerst, et al., (1989) Proc. Natl. Acad. Sci. USA 86:2549-2553; Deuschle, et al., (1990) Science 248:480-483; Gossen (1993) Ph.D. Thesis, University of Heidelberg; Reines, etal., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Baim, et al., (1991) Proc. Natl. Acad. Sci. USA 88:5072-5076; Wyborski, et al., (1991) Nucleic Acids Res. 19:4647-4653; Hillenand-Wissman (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin (1993) Ph.D. Thesis, University of Heidelberg; Gossen, et al., (1992) Proc. Natl. Acad. Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin); Gill, et al., (1988) Nature 334:721-724. Such disclosures are herein incorporated by reference. The above list of selectable marker genes is not meant to be limiting. Any selectable marker gene can be used in the present invention.

The present invention also provides a method for increasing the concentration and/or activity of a VSP polypeptide, for example, the ZmLox6 protein of SEQ ID NO: 2 or biologically active fragment or variant thereof, in a plant. In general, concentration and/or activity is increased by at least 1%, 5%, 10%, 20%, 30%, 40%, 50%, 60%, 70%, 80% or 90% relative to a wild-type or control plant, plant part, or cell that did not have a VSP sequence of the invention introduced. Increasing the concentration and/or activity of a VSP polypeptide in the present invention may occur during and/or subsequent to growth of the plant to the desired stage of development. In specific embodiments, VSP polypeptides such as the ZmLox6 protein or fragment or variant thereof are increased in monocots, including, but not limited to, maize.

The expression level of the VSP polypeptide may be measured directly, for example, by assaying for the level of the VSP polypeptide in the plant.

In specific embodiments, the VSP polypeptide or polynucleotide is introduced into the plant cell. As discussed elsewhere herein, many methods are known in the art for providing a polypeptide to a plant including, but not limited to, direct introduction of the polypeptide into the plant and introducing into the plant (transiently or stably) a polynucleotide construct encoding a polypeptide having VSP properties. Subsequently, a plant cell having the introduced sequence of the invention is selected using methods known to those of skill in the art such as, but not limited to, Southern blot analysis, DNA sequencing, PCR analysis, or phenotypic analysis. A plant or plant part modified by the foregoing embodiments is grown under plant forming conditions for a time sufficient to increase the concentration and/or activity of the VSP polypeptide, for example, the ZmLox6 protein or fragment or variant thereof, in the plant. Plant forming conditions are well known in the art and discussed briefly elsewhere herein.

It is also recognized that the level of the VSP polypeptide may be increased by employing a polynucleotide that is not capable of directing, in a transformed plant, the expression of a protein or an RNA. For example, VSP polynucleotides such as the ZmLox6 gene may be used to design polynucleotide constructs that can be employed in methods for altering or mutating a genomic nucleotide sequence in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such nucleotide constructs and methods of use are known in the art. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham, et al., (1999) Proc. Natl. Acad. Sci. USA 96:8774-8778; herein incorporated by reference. Thus, the level and/or activity of a VSP polypeptide, for example, the ZmLox6 protein of SEQ ID NO: 2 or fragment or variant thereof, may be increased by altering the gene encoding the VSP polypeptide or its promoter. See, e.g., Kmiec, U.S. Pat. No. 5,565,350; Zarling, et al., PCT/US93/03868. Thus mutagenized plants that carry mutations in VSP genes, where the mutations increase expression of the VSP gene, for example, the ZmLox6 gene, or increase the VSP properties of the encoded VSP polypeptide, for example, the ZmLox6 protein, are provided.

It is therefore recognized that methods of the present invention do not depend on the incorporation of an entire polynucleotide into the genome, only that the plant or cell thereof is altered as a result of the introduction of the polynucleotide into a cell. In one embodiment of the invention, the genome may be altered following the introduction of a VSP polynucleotide, such as the ZmLox6 sequence of SEQ ID NO: 1, or the ZmLox6 coding sequence set forth in nucleotides 62-2737 of SEQ ID NO: 1 or in SEQ ID NO: 3, into a cell. For example, the polynucleotide, or any part thereof, may incorporate into the genome of the plant. Alterations to the genome of the present invention include, but are not limited to, additions, deletions, and substitutions of nucleotides into the genome. While the methods of the present invention do not depend on additions, deletions, and substitutions of any particular number of nucleotides, it is recognized that such additions, deletions, or substitutions comprises at least one nucleotide.

Accordingly, in some embodiments, the methods of the invention involve introducing a VSP polypeptide or polynucleotide into a plant. “Introducing” is intended to mean presenting to the plant the VSP polynucleotide or polypeptide in such a manner that the sequence gains access to the interior of a cell of the plant. The methods of the invention do not depend on a particular method for introducing a sequence into a plant, only that the polynucleotide or polypeptide gains access to the interior of at least one cell of the plant. Methods for introducing VSP polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing VSP polypeptides or polynucleotide sequences into plants may vary depending on the type of plant or plant cell targeted for transformation. In some embodiments, the methods of the present invention involve transformation protocols suitable for introducing VSP polypeptides or polynucleotide sequences into monocots.

Suitable methods of introducing VSP polypeptides and polynucleotides into plant cells include microinjection (Crossway, et al., (1986) Biotechniques 4320-334), electroporation (Riggs, et al., (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski, et al., (1984) EMBO J. 3:2717-2722), and ballistic particle acceleration (see, for example, U.S. Pat. No. 4,945,050; U.S. Pat. No. 5,879,918; U.S. Pat. Nos. 5,886,244; and, 5,932,782; Tomes, et al., (1995) in Plant Cell, Tissue, and Organ Culture: Fundamental Methods, ed. Gamborg and Phillips (Springer-Verlag, Berlin); McCabe, et al., (1988) Biotechnology 6:923-926); and Lec1 transformation (WO 00/28058). Also see, Weissinger, et al., (1988) Ann. Rev. Genet. 22:421-477; Sanford, et al., (1987) Particulate Science and Technology 5:27-37 (onion); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783; and, 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp.197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford (1995) Annals of Botany 75:407-413 (rice); Osjoda, et al., (1996) Nature Biotechnology 14:745-750 (maize via Agrobacterium tumefaciens); all of which are herein incorporated by reference.

In specific embodiments, increased nitrogen storage capacity, and concomitant increases in nitrogen content and/or nutritional value, of a plant or plant part thereof, compared to a wild-type or control plant can be provided to a plant using a variety of transient transformation methods. Such transient transformation methods include, but are not limited to, the introduction of the VSP polypeptide, for example, the ZmLox6 protein of SEQ ID NO: 2 or biologically active fragment or variant thereof, directly into the plant or the introduction of a transcript into the plant. Such methods include, for example, microinjection or particle bombardment. See, for example, Crossway, et al., (1986) Mol Gen. Genet. 202:179-185; Nomura, et al., (1986) Plant Sci. 44:53-58; Hepler, et al., (1994) Proc. Natl. Acad. Sci. 91:2176-2180 and Hush, et al., (1994) The Journal of Cell Science 107:775-784, all of which are herein incorporated by reference. Alternatively, a VSP polynucleotide, for example, the ZmLox6 sequence of SEQ ID NO: 1, the ZmLox6 coding sequence set forth in nucleotides 62-2737 of SEQ ID NO: 1 or in SEQ ID NO: 3, or fragment or variant thereof encoding a VSP polypeptide, can be transiently transformed into the plant using techniques known in the art. Such techniques include viral vector systems and the precipitation of the polynucleotide in a manner that precludes subsequent release of the DNA. Thus, the transcription from the particle-bound DNA can occur, but the frequency with which it is released to become integrated into the genome is greatly reduced. Such methods include the use of particles coated with polyethylimine (PEI; Sigma #P3143).

In other embodiments, VSP polynucleotides may be introduced into plants by contacting plants with a virus or viral nucleic acids. Generally, such methods involve incorporating a nucleotide construct within a viral DNA or RNA molecule. It is recognized that a VSP polypeptide of interest may be initially synthesized as part of a viral polyprotein, which later may be processed by proteolysis in vivo or in vitro to produce the desired recombinant protein. Further, it is recognized that useful promoters may include promoters utilized for transcription by viral RNA polymerases. Methods for introducing polynucleotides into plants and expressing a polypeptide encoded thereby, involving viral DNA or RNA molecules, are known in the art. See, for example, U.S. Pat. Nos. 5,889,191; 5,889,190; 5,866,785; 5,589,367; 5,316,931 and Porta, et al., (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO 99/25821, WO 99/25854, WO 99/25840, WO 99/25855, and WO 99/25853, all of which are herein incorporated by reference. Briefly, a polynucleotide can be contained in a transfer cassette flanked by two non-recombinogenic recombination sites. The transfer cassette is introduced into a plant having stably incorporated into its genome a target site that is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick, et al., (1986) Plant Cell Reports 5:81-84. These plants may then be grown, and either pollinated with the same transformed strain or different strains, and the resulting progeny having expression of the desired phenotypic characteristic, for example, increased nitrogen storage capacity, increased nitrogen content, and/or increased nutritional value, identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide described herein, for example, an expression cassette comprising the ZmLox6 sequence of SEQ ID NO: 1, the ZmLox6 coding sequence set forth in nucleotides 62-2737 of SEQ ID NO: 1 or in SEQ ID NO: 3, or fragment or variant thereof encoding a VSP polypeptide, stably incorporated into their genome.

Plants of the invention may be produced by any suitable method, including breeding. Plant breeding can be used to introduce desired characteristics (e.g., a stably incorporated transgene) into a particular plant line of interest, and can be performed in any of several different ways. Pedigree breeding starts with the crossing of two genotypes, such as an elite line of interest and one other elite inbred line having one or more desirable characteristics (i.e., having stably incorporated a polynucleotide of interest, having a modulated activity and/or level of the polypeptide of interest, etc.) which complements the elite plant line of interest. If the two original parents do not provide all the desired characteristics, other sources can be included in the breeding population. In the pedigree method, superior plants are selfed and selected in successive filial generations. In the succeeding filial generations the heterozygous condition gives way to homogeneous lines as a result of self-pollination and selection. Typically in the pedigree method of breeding, five or more successive filial generations of selfing and selection is practiced: F1→F2; F2→F3; F3→F4; F4→F5, etc. After a sufficient amount of inbreeding, successive filial generations will serve to increase seed of the developed inbred. In specific embodiments, the inbred line comprises homozygous alleles at about 95% or more of its loci.

In addition to being used to create a backcross conversion, backcrossing can also be used in combination with pedigree breeding to modify an elite line of interest and a hybrid that is made using the modified elite line. As discussed previously, backcrossing can be used to transfer one or more specifically desirable traits from one line, the donor parent, to an inbred called the recurrent parent, which has overall good agronomic characteristics yet lacks that desirable trait or traits. However, the same procedure can be used to move the progeny toward the genotype of the recurrent parent but at the same time retain many components of the non-recurrent parent by stopping the backcrossing at an early stage and proceeding with selfing and selection. For example, an F1, such as a commercial hybrid, is created. This commercial hybrid may be backcrossed to one of its parent lines to create a BC1 or BC2. Progeny are selfed and selected so that the newly developed inbred has many of the attributes of the recurrent parent and yet several of the desired attributes of the non-recurrent parent. This approach leverages the value and strengths of the recurrent parent for use in new hybrids and breeding.

Therefore, an embodiment of this invention is a method of making a backcross conversion of an inbred line of interest comprising the steps of crossing a plant from the inbred line of interest with a donor plant comprising at least one mutant gene or transgene conferring a desired trait (e.g., increased nitrogen storage capacity), selecting an F1 progeny plant comprising the mutant gene or transgene conferring the desired trait, and backcrossing the selected F1 progeny plant to a plant of the inbred line of interest. This method may further comprise the step of obtaining a molecular marker profile of the inbred line of interest and using the molecular marker profile to select for a progeny plant with the desired trait and the molecular marker profile of the inbred line of interest. In the same manner, this method may be used to produce an F1 hybrid seed by adding a final step of crossing the desired trait conversion of the inbred line of interest with a different plant to make F1 hybrid seed comprising a mutant gene or transgene conferring the desired trait.

In certain embodiments, the monocot-derived VSP polynucleotides of the present invention can be stacked with any combination of polynucleotide sequences of interest in order to create plants with a desired trait. A trait, as used herein, refers to the phenotype derived from a particular sequence or groups of sequences. For example, the VSP polynucleotides of the present invention may be stacked with any other polynucleotides encoding polypeptides having VSP properties, such as an alkaline phosphatase (Dewald, et al., (1992) J. Biol. Chem. 267:15958-15964), amylase (Noquet, et al., (2001) Australian J. Plant Physiol. 28:279-287), chitinase (Peumans, et al., (2002) Plant Physiol. Rockville 130:1063-1072), lectin (Van, et al., (2002) Plant Physiol. Rockville 130:757-769), another lipoxygenase (Tranbarger, et al., (1991) Plant Cell 3:973-988), and the like. The combinations generated can also include multiple copies of any one of the polynucleotides of interest.

The polynucleotides of the present invention can also be stacked with any other gene or combination of genes to produce plants with a variety of desired trait combinations including, but not limited to, traits desirable for animal feed such as high oil genes (e.g., U.S. Pat. No. 6,232,529); balanced amino acids (e.g., hordothionins (U.S. Pat. Nos. 5,990,389; 5,885,801; 5,885,802; and 5,703,409); barley high lysine (Williamson, et al., (1987) Eur. J. Biochem. 165:99-106; and WO 98/20122) and high methionine proteins (Pedersen, et al., (1986) J. Biol. Chem. 261:6279; Kirihara, et al., (1988) Gene 71:359; and Musumura, et al., (1989) Plant Mol. Biol. 12:123)); increased digestibility (e.g., modified storage proteins (U.S. application Ser. No. 10/053,410, filed Nov. 7, 2001); and thioredoxins (U.S. application Ser. No. 10/005,429, filed Dec. 3, 2001)); the disclosures of which are herein incorporated by reference.

The polynucleotides of the present invention can also be stacked with traits desirable for disease or herbicide resistance (e.g., fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (e.g., the EPSPS gene and the GAT gene; see, for example, U.S. Publication No. 20040082770 and WO 03/092360)); and traits desirable for processing or process products such as high oil (e.g., U.S. Pat. No. 6,232,529 ); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)); and polymers or bioplastics (e.g., U.S. Pat. No. 5.602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)); the disclosures of which are herein incorporated by reference. One could also combine the polynucleotides of the present invention with polynucleotides providing agronomic traits such as male sterility (e.g., see U.S. Pat. No. 5,583,210), stalk strength, flowering time, or transformation technology traits such as cell cycle regulation or gene targeting (e.g., WO 99/61619, WO 00/17364, and WO 99/25821); the disclosures of which are herein incorporated by reference.

These stacked combinations can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In one embodiment, it is desirable to introduce a transformation cassette that will result in the overexpression of the polynucleotide of interest. This may be combined with any combination of other overexpression cassettes to generate the desired combination of traits in the plant. It is further recognized that polynucleotide sequences can be stacked at a desired genomic location using a site-specific recombination system. See, for example, WO 99/25821, WO 99/25854, WO 99/25840, WO 99/25855, and WO 99/25853, all of which are herein incorporated by reference.

As used herein, the term “plant” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides. Thus, the invention provides transgenic seeds produced by the plants of the invention.

A “subject plant or plant cell” is one in which a genetic alteration, such as transformation, has been effected as to a VSP gene of interest, or is a plant or plant cell that is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct that has no known effect on the trait of interest, such as a construct comprising a marker gene); (c) a plant or plant cell that is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the VSP gene of interest is not expressed.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.).

In other embodiments, plants of interest are monocots, for example, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum aestivum), sugarcane (Saccharum spp.), oats, and barley.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

The following examples are offered by way of illustration and not by way of limitation.

### EXPERIMENTAL

Plants are known to accumulate VSPs as a mechanism to sequester excess nitrogen in their vegetative cells, particularly when a reproductive sink is limiting (Staswick (1994) Ann. Rev. Plant Physiol. Plant Mol. Biol. 45:303-322). The leaves of the deciduous trees recycle their nitrogen before they are shed in autumn. The recycled nitrogen is stored in the bark in the form of VSPs. The VSPs are remobilized when the demand for nitrogen exceeds the amount available in the cell, e.g., during reproductive sink development or during spring growth (Staswick (1994) Ann. Rev. Plant Physiol. Plant Mol. Biol. 45:303-322). VSPs, ranging in size from ˜15 to ˜100 kDa, have been identified as an alkaline phosphatase (Dewald, et al., (1992) J. Biol. Chem. 267:15958-15964), amylase (Noquet, et al., (2001) Australian J. Plant Physiol. 28:279-287), chitinase (Peumans, et al., (2002) Plant Physiol. 130:1063-1072), lectin (Van, et al., (2002) Plant Physiol. 130:757-769), or a lipoxygenase (Tranbarger, et al., (1991) Plant Cell 3:973-988). Their occurrence has been reported in a wide variety of annual and perennial plant species: soybean (Staswick (1988) Plant Physiol. 87:250-254; Tranbarger, et al., (1991) Plant Cell 3:973-988); Trifolium (Corre, et al., (1996) J. Exp. Botany 47:1111-1118); Medicago (alfalfa) (Avice, et al., (1997) Crop Sci. 37:1187-1193; Noquet, et al., (2001) Australian J. Plant Physiol. 28:279-287); Arabidopsis (Utsugi, et al., (1998) Plant Mol. Biol. 38:565-576); canola (Rossato, et al., (2002) J. Exp. Botany 53:265-275); poplar (Lawrence, et al., (1997) Planta Heidelberg 203:237-244); black mulberry (Van, et al., (2002) Plant Physiol. 130:757-769); and peach (Gomez & Faurobert (2002) J. Exp. Botany 53:2431-2439). However, occurrence of VSPs in monocots has not heretofore been established (Mackown, et al., (1992) Plant Physiol. 99:1469-1474).

Proteins known to be a VSP in one species can also be expressed at high levels in another species where they are not normally expressed. For example, the transgenically expressed soybean VSP accumulated to a level of ˜5% of the soluble proteins in tobacco (Guenoune, et al., (1999) Plant Science 145:93-98; Guenoune, et al., (2002) J. Exp. Botany 53:1867-1870). Different VSP proteins may employ different mechanisms for intracellular targeting. For example, VSP-alpha follows the ER-Golgi path for targeting to the vacuole, whereas lipoxygenase (Lox), also known as a VLX (vegetative lipoxygenase), follows a different, unknown path to the vacuolar compartment (Klauer and Franceschi (1997) Protoplasma 200:174-185). Different VLX proteins accumulate in separate intracellular compartments in soybean: VLX A, B, and C accumulate in the cytosol; VLX D is sequestered in the vacuole of the bundle-sheath and paraveinal cells (Fischer, et al., (1999) Plant Journal 19:543-554). The VLX proteins accumulate even under low N, however, suggesting that they play a broader role than just as VSPs (Grimes, et al., (1993) Plant Physiol. 103:457-466).

Plants apparently perceive stress as a signal for tissue and thus nitrogen loss. To account for this, VSPs are known to accumulate when plants are exposed to water stress and methyl jasmonate, a stress hormone (Mason and Mullet (1990) Plant Cell 2:569-580; Rossato, et al., (2002) J. Exp. Botany 53:1131-1141). Other stresses, such as wounding, herbivore damage, senescence, and ozone are also known to lead to their accumulation (Utsugi, et al., (1998) Plant Mol. Biol. 38:565-576; Berger, et al., (2002) Physiologia Plantarum 114:85-91; Mira, et al., (2002) Planta Berlin 214:939-946).

The present examples focus on one lipoxygenase gene out of eleven in maize that exhibits the characteristics of a VSP. Results demonstrate that the maize lipoxygenase ZmLox6 is induced upon supplying high levels of N in the growth medium and is most highly expressed in the leaves, just like the soybean VSP VLX D (Tranbarger, et al., (1991) Plant Cell 3:973-988).

### EXAMPLE 1

### Induction of Proteins by Nitrogen in the Growth Medium

Corn seedlings were tested for the induction of proteins by either nitrate or a combination of nitrate and ammonium in the growth medium. Two-week-old plants grown in vermiculite in the greenhouse in the absence of applied nitrogen showed signs of nitrogen deficiency as judged from the yellowing of the leaves. Some yellowing of the leaves was observed even at 1 mM nitrate in the growth medium. In order to identify the nitrogen-inducible proteins, excessive amounts of nitrogen were supplied in the growth medium to induce expression of proteins associated with any endogenous nitrogen storage machinery. Upon application of a 100 mM nitrate-only source of nitrogen, stress (leaf rolling) symptoms were obvious. When supplied with 50 mM ammonium nitrate (100 mM total nitrogen), the plants looked healthier than at 1 mM or 100 mM nitrate. Ammonium nitrate treatment was included to determine if any different proteins were induced relative to nitrate treatment alone.

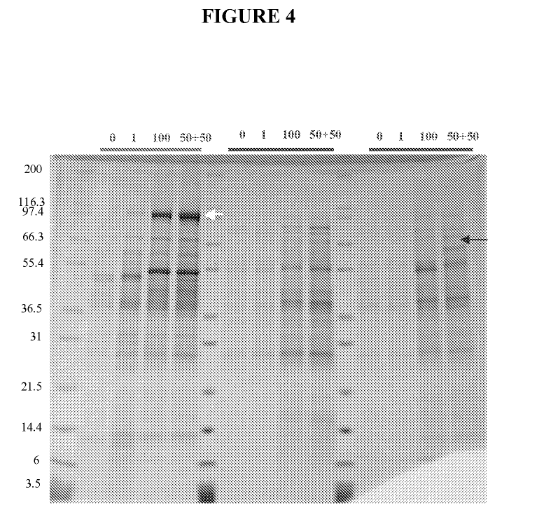

Different tissues from the plants grown at different nitrogen levels were homogenized in a buffer solution and centrifuged at 100,000× g in an ultracentrifuge. Both the pellet and the supernatant were subjected to SDS-PAGE. A polypeptide band at ˜100 kDa was strongly induced in the soluble fraction at higher levels of nitrogen (see FIG. 4). The induction was strongest in the leaf tissue. This polypeptide was undetectable in the root tissue. Another polypeptide of ˜60 kDa appeared to be induced in the stem tissue when ammonium nitrate was supplied as a source of nutrition.

### EXAMPLE 2

### Protein Processing for Proteomic Analysis

The 98 kDa protein band from the soluble leaf protein fraction in Example 1 whose expression level increased with increasing nitrate supplementation was excised from a Tris-glycine-SDS gel and minced coarsely. Gel pieces (approximately 200 μL volume) were washed in 500 μL of 100 mM ammonium bicarbonate, then gradually dehydrated in increasing acetonitrile % (15%, 50%, 100%). Dried gel pieces were rehydrated on ice for 1 hr in 250 μL of trypsin (Roche 1418025) solution containing approximately 4 μg trypsin in 15% acetonitrile/100 mM ammonium bicarbonate. Unabsorbed fluid was aspirated and saved at 4° C. 200 μL buffer was added, and in-gel digestion proceeded for 16 hr at 37° C. Gel pieces were washed in 200 μL of 15% acetonitrile/100 mM ammonium bicarbonate for 30 min at 37° C., and fluid collected and pooled. Proteolytic peptides were collected by washing the gel pieces in increasing acetonitrile % (15%, 50% and 100%), and pooling aspirated fluid. The pooled aspirant was dried completely under vacuum, and the residue redissolved in 20 μL H₂O containing 0.1% formic acid. The entire sample was injected into a 1 μL loop and the peptides were subsequently trapped on a polymeric trap column. Reversed-phase chromatography was performed using a C18 silica column, 75 μm×100 mm, at a flow rate of 200 nL/min with an acetonitrile gradient of 3-85%. A repeating data-dependent MS experiment was set up on an LCQ Classic quadrapole ion trap mass spectrometer to acquire one full scan MS followed by three MS/MS scans of the most abundant precursor ions for the duration of the run. The acquired data were then searched using Sequest software to identify sequence information for the individual peptide fragments.

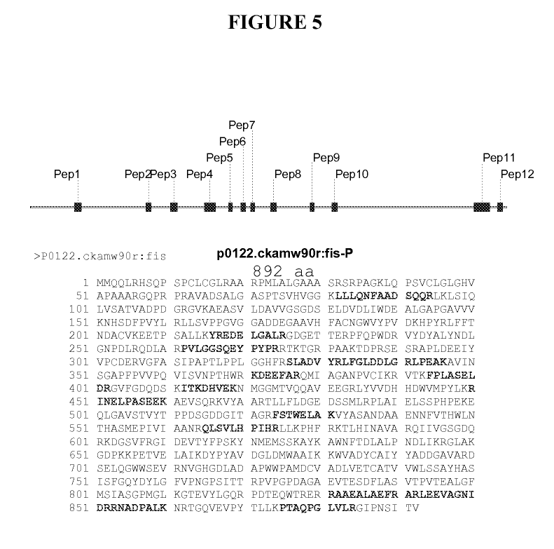

Twelve different peptides belonging to the same lipoxygenase polypeptide (ZmLox6) were identified (see FIG. 5). The coverage is all over the protein, strongly indicating that the identified protein is indeed ZmLox6.

In addition to the ZmLox6, phosphoenolpyryvate carboxylase (PEP carboxylase, ˜110 kDa), pyruvate orthophosphate dikinase (PPDK, Mr ˜120 kDa), aconitate hydratase C (ACH, Mr ˜116 kDa), and a putative protein that has been tentatively annotated as a cell division protein (Mr ˜90 kDa) were induced by high N in the growth medium. Apparently, the predicted 90 kDa protein was glycosylated as it migrated as a >100 kDa protein. The first three enzymes, PEP carboxylase, PPDK and ACH, are all C4 enzymes.

### EXAMPLE 3

### Phylogenetic Analysis of ZmLox6 with Other Proteins

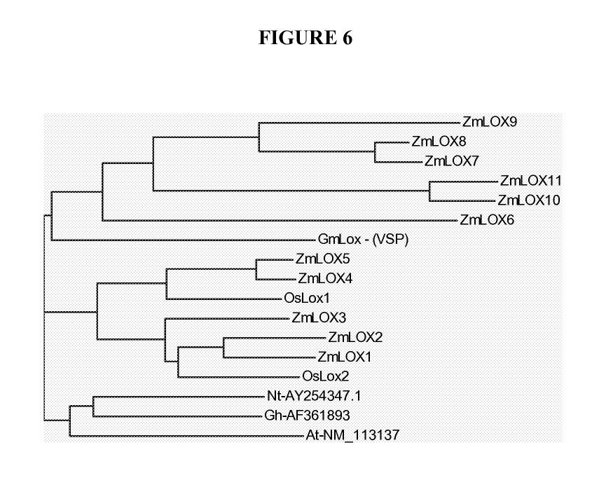

Upon BLAST analysis against public databases, ZmLox6 protein shows highest homology (43% identity, 57% similarity) with the rice Lox1 protein. Without being bound by theory, this rather low homology suggests that ZmLox6 has evolved independently to perhaps carry out some species-specific function. Upon phylogenetic analysis using Lox proteins from several other plant species as well as from maize, the ZmLox6 protein was found to be closest to the soybean Lox protein (see FIG. 6). The soybean Lox protein has been previously demonstrated to be a vegetative storage protein that accumulates in the vacuoles of the mesophyll cells surrounding the veins in the leaves (Tranbarger, et al., (1991) Plant Cell 3:973-988). These results suggest that the ZmLox6 protein may also be a vegetative storage protein that may have an orthologous function to that of the soybean Lox protein.

### EXAMPLE 4

### Nitrogen-Induced Proteins Accumulate Most Highly in Fully Expanded Leaves

Proteins from individual leaves collected from 16-day-old maize plants grown in either 0.1 mM or 50 mM NH₄NO₃ were subjected to SDS-PAGE in order to identify the leaves with highest expression of the polypeptide band at ˜100-110 kDa. The polypeptide band at ˜100 kDa was most abundant in leaf 4, which was fully expanded as judged from the lack of light green basal portion and the lack of any senescent parts as seen in older leaves 1, 2 and 3. Although it is unclear what proportion of this band could be accounted for by ZmLox6, it is quite clear that the proteins in this band were not present to any appreciable extent in younger leaves 7 and 8. This variation is consistent with the hypothesis that cells would sequester nitrogen into a VSP only when excess of it is available, a scenario likely to occur in fully expanded leaves but not in the young, rapidly expanding ones.

### EXAMPLE 5

### Expression Pattern of ZmLox6 as Studied by Lynx MPSS

The expression pattern of maize Lox genes in different tissues of the inbred line A63 was compiled from the MPSS database. The number of libraries sampled for each tissue were as follows: meristem, 14; root, 33; stalk, 11; leaf, 35; ear, 15; husk, 1; whole kernel, 2; embryo, 8; endosperm, 19; pericarp, 6; silk, 7; tassel, 14; anther, 2; pollen, 1. As shown in Table 1, although expressed at a lower level in a number of tissues, ZmLox6 is most highly expressed in the leaf tissue.

TABLE 1
Expression pattern of maize Lox genes.

| Tissue | Lox1 | Lox2 | Lox3 | Lox4 | Lox5 | Lox6 | Lox7 | Lox8 | Lox9 | Lox10 | Lox11 |
|---|---|---|---|---|---|---|---|---|---|---|---|
| meristem | 46 | 119 | 17 | 35 | 243 | 59 | 0 | 0 | 21 | 157 | 1 |
| root | 2065 | 848 | 675 | 303 | 162 | 114 | 0 | 0 | 21 | 423 | 35 |
| stalk | 395 | 1557 | 9 | 55 | 567 | 190 | 0 | 0 | 18 | 880 | 21 |
| leaf | 195 | 98 | 42 | 35 | 166 | 1312 | 0 | 0 | 22 | 5851 | 13 |
| ear | 3 | 311 | 2 | 68 | 260 | 0 | 0 | 0 | 1 | 50 | 5 |
| husk | 193 | 2523 | 28 | 161 | 433 | 0 | 0 | 0 | 0 | 1480 | 4 |
| kernel | 146 | 2701 | 140 | 63 | 613 | 0 | 0 | 0 | 0 | 1215 | 9 |
| embryo | 1 | 15 | 125 | 36 | 23 | 0 | 0 | 0 | 0 | 10 | 0 |
| endosperm | 1 | 8 | 857 | 19 | 9 | 2 | 0 | 2 | 2 | 2 | 8 |
| pericarp | 7 | 476 | 783 | 24 | 195 | 108 | 0 | 0 | 3 | 18 | 8 |
| silk | 0 | 226 | 42 | 22 | 800 | 0 | 0 | 0 | 0 | 1447 | 3 |
| tassel | 32 | 577 | 17 | 46 | 800 | 1 | 0 | 0 | 0 | 684 | 18 |
| anther | 282 | 0 | 534 | 38 | 14 | 83 | 0 | 0 | 9 | 110 | 0 |
| pollen | 0 | 3 | 0 | 24 | 0 | 0 | 0 | 0 | 0 | 0 | 0 |

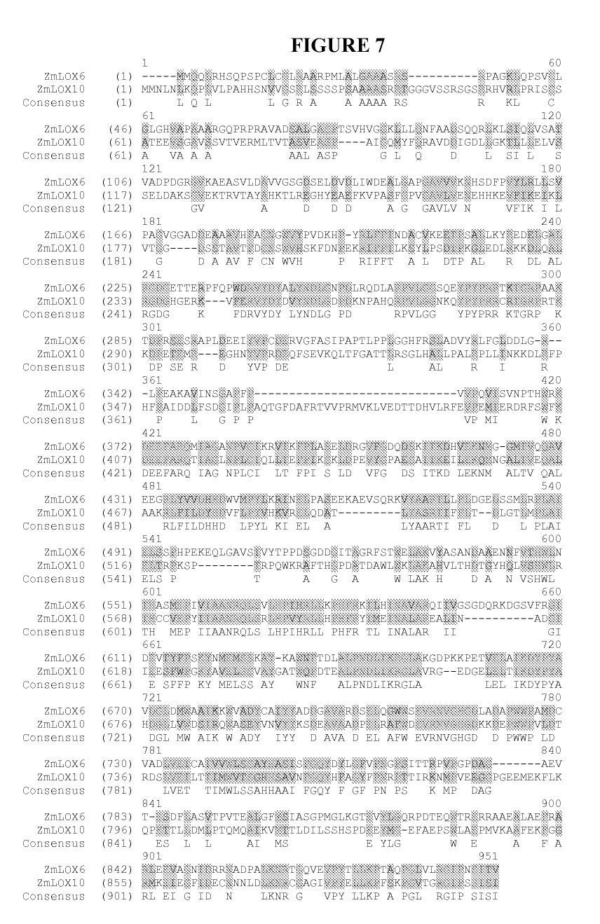

Another gene that is highly expressed in the leaf tissue is ZmLox10. However, not a single peptide for the protein encoded by ZmLox10 was detected during proteomics analysis of the nitrogen-inducible polypeptide band from the leaf tissue (see Examples 1 and 2). The predicted molecular masses of ZmLox6 (amino acid sequence shown in SEQ ID NO: 2) and ZmLox10 (amino acid sequence shown in SEQ ID NO: 4) are approximately 97 and 102 kDa, respectively, and the two polypeptides share only 34% identity (see FIG. 7). The two proteins are sufficiently different that if the ZmLox10 were present at a detectable level in the ˜100 kDa polypeptide band, it could have been picked up by the proteomics analysis. This suggests that ZmLox10 was not induced under the experimental conditions used, leaving ZmLox6 as the only VSP-like protein.

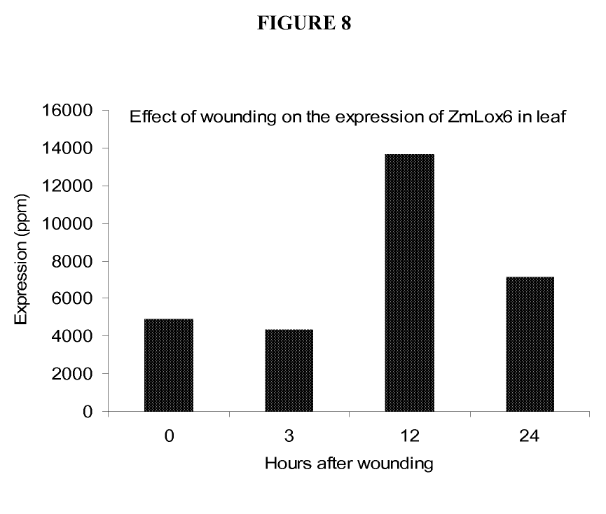

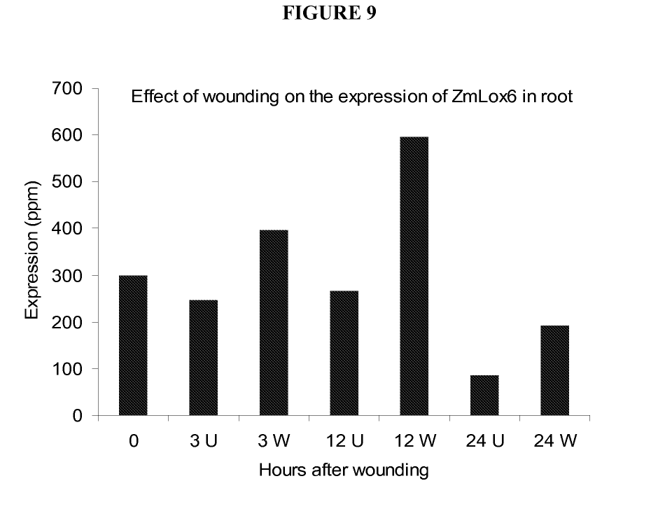

Induction of expression of the ZmLox6 gene following wounding was then studied in the V5 corn leaf and in the corn nodal root at V5 stage of development. Induction of expression was measured in ppm over time at 0, 3, 12 and 24 hours following wounding. Results showed that ZmLox6 was induced by wounding in both the leaf as well as the root tissue (see FIGS. 8 and 9), a characteristic exhibited by VSPs from other plant species (Utsugi, et al., (1998) Plant Mol. Biol. 38:565-576; Berger, et al., (2002) Physiologia Plantarum 114:85-91; Mira, et al., (2002) Planta Berlin 214:939-946).

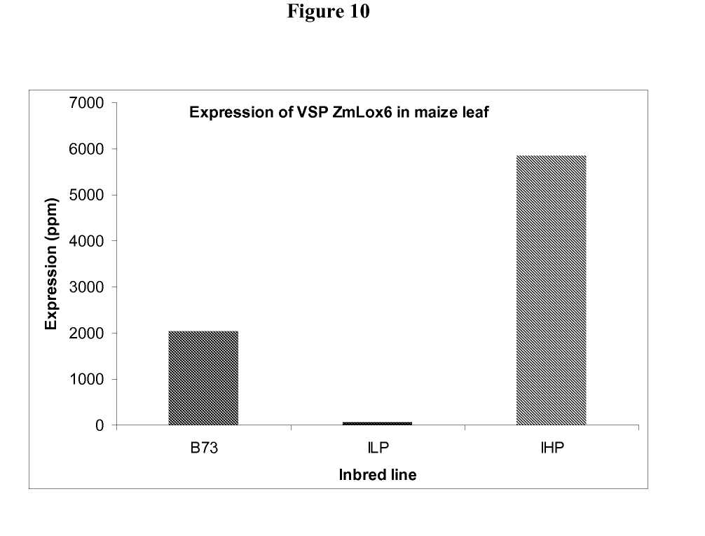

Illinois high protein (IHP) and Illinois low protein (ILP) lines have been selected over a hundred cycles for high or low grain protein, respectively (Uribelarrea, et al., (2004) Crop Science 44:1593-1600). Whereas IHP grains contain >25% protein, those of ILP have <5%. The high demand for nitrogen in the grain of IHP is met by a greater amount of nitrogen in its vegetative tissues since it is well known that most of the nitrogen in the vegetative tissues is remobilized to grain by maturity. MPSS analysis of these lines revealed that ZmLox6 was expressed at a very low level in ILP in comparison to that in IHP, implying the role of this protein in nitrogen storage in the vegetative tissues (FIG. 10).

Collectively, these findings support the results described above from nitrogen-induction and proteomics studies, suggesting that ZmLox6 is a VSP in corn and is highly expressed in the leaf tissue.

### EXAMPLE 6

### Expression of ZmLox6 in E. coli

Full-length ZmLox6 was amplified from an expressed-sequence-tagged clone by PCR to generate an in-frame EcoRI restriction site upstream of the ATG, and an in-frame XhoI restriction site immediately following the coding sequence, to produce a product of 2,676 bp. Amplification primer sequences: upstream, 5′-GTTACCGAATTCGCCCTTCCCGGTACCATGATG-3′ (SEQ ID NO: 5) and downstream, 5′-CGCCTCCCTCGAGAACGGTGAGGCTGTTG-3′ (SEQ ID NO: 6). PCR product band was excised from an ethidium-stained 0.5× TBE agarose gel, eluted using Bio-Rad's “Freeze & Squeeze” spin columns, and digested with EcoRI+XhoI overnight. Restricted PCR product was purified from the reaction mix using a QiaQuick spin-column (Qiagen), and concentrated by evaporation under vacuum. Expression vector pET-28a (Novagen) was digested overnight with EcoRI+XhoI, and gel-purified, eluted, and concentrated as described above. Ligation and transformation were performed using standard protocols as supplied from the manufacturers (Rapid DNA Ligation Kit from Roche; One Shot Chemically Competent TOP10 Cells from Invitrogen). Plasmid DNA from kanamycin-resistant colonies was analyzed by EcoRI-XhoI restriction to verify presence of cloned ZmLox6.

pET-28a/ZmLox6 vector was transformed into expression host Rosetta (DE3)pLacl (Novagen) using the supplier's standard protocol. Chloramphenicol- and kanamycin-resistant transformants were screened by IPTG-induced protein expression in 2-mL test cultures. One high-expressing transformant was selected for solubility studies. Cell lysis and solubilization were achieved using the following detergent lysis buffer: 50 mM sodium phosphate pH 7.7, 2% (w/v) Triton X-100, ±200 μg/mL lysozyme. Recombinant ZmLox6 protein was found to accumulate in the insoluble inclusion bodies, and was only partially liberated from this fraction with 8 M urea.

Expression cultures were scaled up to 2 L (4×500 mL). Cells were pelleted and frozen at −80° C. Thawed cell pellets were resuspended in lysis buffer by pipetting, then vigorous vortexing. Lysates were pelleted and again resuspended in lysis buffer with lysozyme. An excess of 1:10 dilution lysis buffer was added, and insoluble lysate pelleted. The insoluble lysate was resuspended in 1:10 dilution lysis buffer as above, and inclusion bodies collected by centrifugation. Inclusion bodies were washed once in 1:10 dilution lysis buffer and re-pelleted. Purified inclusion body pellets were solubilized directly in LiDS sample buffer by pipetting, heated to 100° C., and run on Tris-glycine 10% acrylamide preparative gels. Gels were washed extensively in pure water and stained very briefly in aqueous Coomassie (SimplyBlue Safe Stain, Invitrogen). Recombinant ZmLox6 protein resolved as a broad band between 95-98 kDa (see FIG. 10; SeeBlue Plus 2 MW markers, Invitrogen). Bands were excised from 24 preparative gels; protein was electroeluted (Elutrap, Schleicher & Schuell) and concentrated/desalted (Centriprep spin columns, 3,000 MWCO, Millipore). Total recovery, as estimated from in-gel comparison with stained BSA standards, was approximately 2 mg.

### EXAMPLE 7

### Production of Anti-ZmLox6 Antibody and Its Use to Study Expression and Localization of This Protein

The electroeluted protein was injected into rabbits to raise antisera as mainly as previously described (Dhugga and Ray (1994) Eur. J. Biochem. 220:943-953) through Strategic Biosolutions (www.strategicbiosolutions.com). The antibody so generated recognized a single polypeptide band of ˜100 kDa on protein blots of maize leaf extracts at an antibody dilution 500,000-fold.

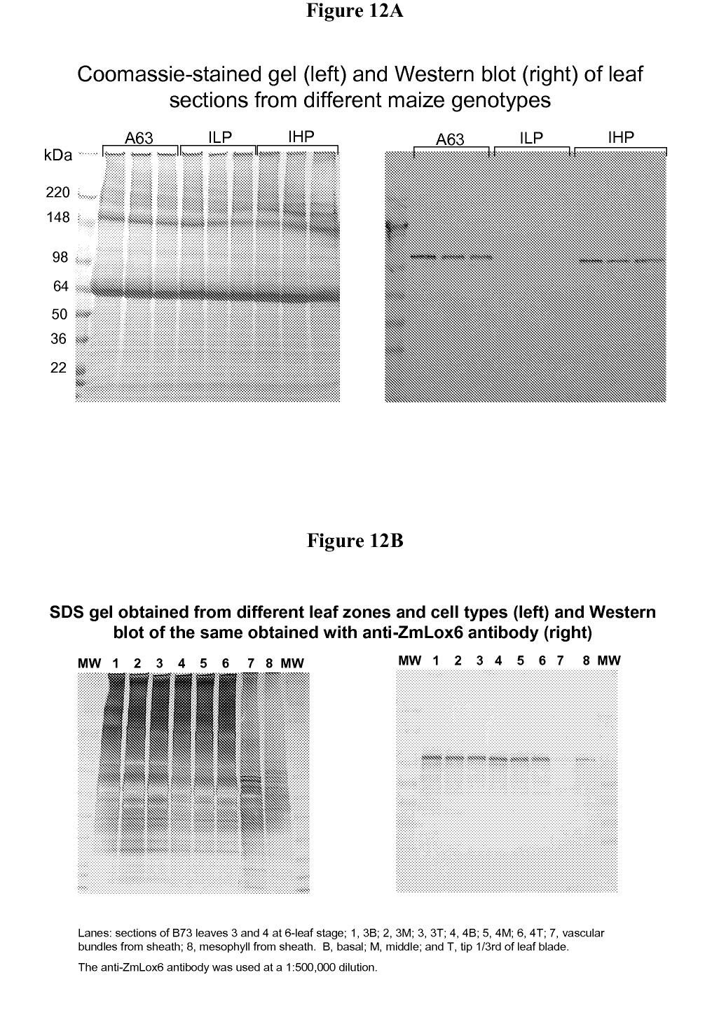

When the leaf extracts from B73, IHP, and ILP were probed with this antibody, results strikingly similar to those found in gene expression analysis were observed, with very low level of protein expression in the IHP leaves (FIGS. 10 and 12A).

To determine the cell-type localization of ZmLox6, the leaf sheaths from the same leaves as used to do Western analysis above were dissected into vascular bundles and mesophyll layers. Western blot analysis using the anti-ZmLox6 antibody of the protein blots derived from these tissues revealed that this protein was expressed in the mesophyll cells and not the vascular bundles (FIG. 12B).

### EXAMPLE 8

### Transformation and Regeneration of Transgenic Plants

Immature maize embryos from greenhouse donor plants are bombarded with a plasmid containing the ZmLox6 sequence of SEQ ID NO: 1 or the ZmLox6 coding sequence of SEQ ID NO: 3 operably linked to the maize Rubisco small subunit (SSU) promoter (FIG. 1A), maize phosphoenolpyruvate carboxylase (PEPC1) promoter (FIG. 2A), or maize ubiquitin-1 (UBI1) promoter (FIG. 3A), and the selectable marker gene PAT (Wohlleben, et al., (1988) Gene 70:25-37), which confers resistance to the herbicide Bialaphos. Alternatively, the selectable marker gene is provided on a separate plasmid.

The construct shown in FIG. 1A provides for preferential expression of the encoded VSP within the bundle-sheath cells of the maize leaf tissues. Alternatively, this construct further comprises a coding sequence for the maize proaleurain vacuolar sorting signal operably linked to the VSP polynucleotide (see FIG. 1B) so that the expressed VSP is directed to the vacuolar compartment of the bundle-sheath cells.

The construct shown in FIG. 2A provides for preferential expression of the encoded VSP within the mesophyll cells of the maize leaf tissue. Alternatively, this construct further comprises a coding sequence for the maize proaleurain vacuolar sorting signal operably linked to the VSP polynucleotide (see FIG. 2B) so that the expressed VSP is directed into the vacuolar compartment of the mesophyll cells, or a coding sequence for a plastid transit peptide, for example, a chloroplast transit peptide, operably linked to the VSP polynucleotide so that the expressed VSP is directed into the plastid compartment of the mesophyll cells.

The construct shown in FIG. 3A provides for constitutive expression of the encoded VSP. Alternatively, this construct further comprises the maize proaleurain vacuolar sorting signal operably linked to the VSP polynucleotide (FIG. 3B) so that the expressed VSP is directed into the vacuolar compartment of the cells in which it is constitutively expressed.

Transformation is performed as follows. Media recipes follow below.

Preparation of Target Tissue

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

The plasmid vector of choice shown in FIG. 1A, 1B, 2A, 2B, 3A or 3B is made. This plasmid DNA is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂; and 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 μl 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #4 in a particle gun. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for total nitrogen content (whole plant and leaf, stem, and seed).

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos(both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### EXAMPLE 9

### Agrobacterium-Mediated Transformation

For Agrobacterium-mediated transformation of maize with a nucleotide sequence comprising the ZmLox6 sequence set forth in SEQ ID NO: 1, the ZmLox6 coding sequence set forth in SEQ ID NO: 3, or a nucleotide sequence that encodes the ZmLox6 protein set forth in SEQ ID NO: 2, the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the nucleotide sequence comprising the sequence set forth in SEQ ID NO: 1, the ZmLox6 coding sequence set forth in SEQ ID NO: 3, or a nucleotide sequence that encodes the ZmLox6 protein set forth in SEQ ID NO: 2 to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants.

### EXAMPLE 10

### Soybean Embryo Transformation

Culture Conditions

Soybean embryogenic suspension cultures (cv. Jack) are maintained in 35 ml liquid medium SB196 (see recipes below) on rotary shaker, 150 rpm, 26° C. with cool white fluorescent lights on 16:8 hr day/night photoperiod at light intensity of 60-85 μE/m2/s. Cultures are subcultured every 7 days to two weeks by inoculating approximately 35 mg of tissue into 35 ml of fresh liquid SB196 (the preferred subculture interval is every 7 days).

Soybean embryogenic suspension cultures are transformed with the plasmids and DNA fragments described in the following examples by the method of particle gun bombardment (Klein, et al., (1987) Nature, 327:70).

Soybean Embryogenic Suspension Culture Initiation

Soybean cultures are initiated twice each month with 5-7 days between each initiation.

Pods with immature seeds from available soybean plants 45-55 days after planting are picked, removed from their shells and placed into a sterilized magenta box. The soybean seeds are sterilized by shaking them for 15 minutes in a 5% Clorox solution with 1 drop of ivory soap (95 ml of autoclaved distilled water plus 5 ml Clorox and 1 drop of soap). Mix well. Seeds are rinsed using 2 1-liter bottles of sterile distilled water and those less than 4 mm are placed on individual microscope slides. The small end of the seed is cut and the cotyledons pressed out of the seed coat. Cotyledons are transferred to plates containing SB1 medium (25-30 cotyledons per plate). Plates are wrapped with fiber tape and stored for 8 weeks. After this time secondary embryos are cut and placed into SB196 liquid media for 7 days.

Preparation of DNA for Bombardment

Either an intact plasmid or a DNA plasmid fragment containing the ZmLox6 sequence set forth in SEQ ID NO: 1, the ZmLox6 coding sequence set forth in SEQ ID NO: 3, or a nucleotide sequence that encodes the ZmLox6 protein set forth in SEQ ID NO: 2 operably linked to the promoter of interest and the selectable marker gene are used for bombardment. Plasmid DNA for bombardment are routinely prepared and purified using the method described in the Promega™ Protocols and Applications Guide, Second Edition (page 106). Fragments of the plasmids carrying the ZmLox6 sequence set forth in SEQ ID NO: 1, the ZmLox6 coding sequence set forth in SEQ ID NO: 3, or a nucleotide sequence that encodes the ZmLox6 protein set forth in SEQ ID NO: 2 operably linked to the promoter of interest and the selectable marker gene are obtained by gel isolation of double digested plasmids. In each case, 100 μg of plasmid DNA is digested in 0.5 ml of the specific enzyme mix that is appropriate for the plasmid of interest. The resulting DNA fragments are separated by gel electrophoresis on 1% SeaPlaque GTG agarose (BioWhitaker Molecular Applications), and the DNA fragments containing the ZmLox6 sequence set forth in SEQ ID NO: 1, the ZmLox6 coding sequence set forth in SEQ ID NO: 3, or a nucleotide sequence that encodes the ZmLox6 protein set forth in SEQ ID NO: 2 operably linked to the promoter of interest and the selectable marker gene are cut from the agarose gel. DNA is purified from the agarose using the GELase digesting enzyme following the manufacturer's protocol.

A 50 μl aliquot of sterile distilled water containing 3 mg of gold particles is added to 5 μl of a 1 μg/μl DNA solution (either intact plasmid or DNA fragment prepared as described above), 50 μl 2.5M CaCl₂ and 20 μl of 0.1 M spermidine. The mixture is shaken 3 min on level 3 of a vortex shaker and spun for 10 sec in a bench microfuge. After a wash with 400 μl 100% ethanol the pellet is suspended by sonication in 40 μl of 100% ethanol. Five μl of DNA suspension is dispensed to each flying disk of the Biolistic PDS1000/HE instrument disk. Each 5 μl aliquot contains approximately 0.375 mg gold per bombardment (i.e., per disk).

Tissue Preparation and Bombardment with DNA

Approximately 150-200 mg of 7 day old embryonic suspension cultures are placed in an empty, sterile 60×15 mm petri dish and the dish covered with plastic mesh. Tissue is bombarded 1 or 2 shots per plate with membrane rupture pressure set at 1100 PSI and the chamber evacuated to a vacuum of 27-28 inches of mercury. Tissue is placed approximately 3.5 inches from the retaining/stopping screen.

Selection of Transformed Embryos

Transformed embryos are selected either using hygromycin (when the hygromycin phosphotransferase, HPT, gene is used as the selectable marker) or chlorsulfuron (when the acetolactate synthase, ALS, gene is used as the selectable marker).

Hygromycin (HPT) Selection

Following bombardment, the tissue is placed into fresh SB196 media and cultured as described above. Six days post-bombardment, the SB196 is exchanged with fresh SB196 containing a selection agent of 30 mg/L hygromycin. The selection media is refreshed weekly. Four to six weeks post-selection, green, transformed tissue may be observed growing from untransformed, necrotic embryogenic clusters. Isolated, green tissue is removed and inoculated into multiwell plates to generate new, clonally propagated, transformed embryogenic suspension cultures.

Chlorsulfuron (ALS) Selection

Following bombardment, the tissue is divided between 2 flasks with fresh SB196 media and cultured as described above. Six to seven days post-bombardment, the SB196 is exchanged with fresh SB196 containing selection agent of 100 ng/ml Chlorsulfuron. The selection media is refreshed weekly. Four to six weeks post-selection, green, transformed tissue may be observed growing from untransformed, necrotic embryogenic clusters. Isolated, green tissue is removed and inoculated into multiwell plates containing SB196 to generate new, clonally propagated, transformed embryogenic suspension cultures.

Regeneration of Soybean Somatic Embryos Into Plants

In order to obtain whole plants from embryogenic suspension cultures, the tissue must be regenerated.

Embryo Maturation

Embryos are cultured for 4-6 weeks at 26° C. in SB196 under cool white fluorescent (Phillips cool white Econowatt F40/CW/RS/EW) and Agro (Phillips F40 Agro) bulbs (40 watt) on a 16:8 hr photoperiod with light intensity of 90-120 μE/m²s. After this time embryo clusters are removed to a solid agar media, SB166, for 1-2 weeks. Clusters are then subcultured to medium SB103 for 3 weeks. During this period, individual embryos can be removed from the clusters and screened for increased nitrogen content compared to wild-types or controls. It should be noted that any detectable phenotype, resulting from the expression of the genes of interest, could be screened at this stage.

Embryo Desiccation and Germination

Matured individual embryos are desiccated by placing them into an empty, small petri dish (35×10 mm) for approximately 4-7 days. The plates are sealed with fiber tape (creating a small humidity chamber). Desiccated embryos are planted into SB71-4 medium where they were left to germinate under the same culture conditions described above. Germinated plantlets are removed from germination medium and rinsed thoroughly with water and then planted in Redi-Earth in 24-cell pack tray, covered with clear plastic dome. After 2 weeks the dome is removed and plants hardened off for a further week. If plantlets looked hardy they are transplanted to 10″ pot of Redi-Earth with up to 3 plantlets per pot. After 10 to 16 weeks, mature seeds are harvested, chipped and analyzed for proteins.

Media Recipes
SB 196 - FN Lite liquid proliferation medium (per liter) -

| | | |
|---|---|---|
| MS FeEDTA - 100× Stock 1 | 10 | ml |
| MS Sulfate - 100× Stock 2 | 10 | ml |
| FN Lite Halides - 100× Stock 3 | 10 | ml |
| FN Lite P, B, Mo - 100× Stock 4 | 10 | ml |
| B5 vitamins (1 ml/L) | 1.0 | ml |
| 2,4-D (10 mg/L final concentration) | 1.0 | ml |
| KNO₃ | 2.83 | gm |
| (NH₄)₂SO₄ | 0.463 | gm |
| Asparagine | 1.0 | gm |
| Sucrose (1%) | 10 | gm |
| pH | 5.8 | |

FN Lite Stock Solutions

| Stock # | | 1000 ml | 500 ml |
|---|---|---|---|
| 1 | MS FeEDTA 100× Stock | | |
| | Na₂ EDTA* | 3.724 g | 1.862 g |
| | FeSO₄—7H₂O | 2.784 g | 1.392 g |
| 2 | MS Sulfate 100× stock | | |
| | MgSO₄—7H₂O | 37.0 g | 18.5 g |
| | MnSO₄—H₂O | 1.69 g | 0.845 g |
| | ZnSO₄—7H₂O | 0.86 g | 0.43 g |
| | CuSO₄—5H₂O | 0.0025 g | 0.00125 g |
| 3 | FN Lite Halides 100× Stock | | |
| | CaCl₂—2H₂O | 30.0 g | 15.0 g |
| | KI | 0.083 g | 0.0715 g |
| | CoCl₂—6H₂O | 0.0025 g | 0.00125 g |
| 4 | FN Lite P, B, Mo 100× Stock | | |
| | KH₂PO₄ | 18.5 g | 9.25 g |
| | H₃BO₃ | 0.62 g | 0.31 g |
| | Na₂MoO₄—2H₂O | 0.025 g | 0.0125 g |

*Add first, dissolve in dark bottle while stirring

SB1 solid medium (per liter) comprises: 1 pkg. MS salts (Gibco/BRL—Cat# 11117-066); 1 ml B5 vitamins 1000× stock; 31.5 g sucrose; 2 ml 2,4-D (20 mg/L final concentration); pH 5.7; and, 8 g TC agar.

SB 166 solid medium (per liter) comprises: 1 pkg. MS salts (Gibco/BRL—Cat# 11117-066); 1 ml B5 vitamins 1000× stock; 60 g maltose; 750 mg MgCl₂ hexahydrate; 5 g activated charcoal; pH 5.7; and, 2 g gelrite.

SB 103 solid medium (per liter) comprises: 1 pkg. MS salts (Gibco/BRL—Cat# 11117-066); 1 ml B5 vitamins 1000× stock; 60 g maltose; 750 mg MgCl₂ hexahydrate; pH 5.7; and, 2 g gelrite.

SB 71-4 solid medium (per liter) comprises: 1 bottle Gamborg's B5 salts w/sucrose (Gibco/BRL—Cat# 21153-036); pH 5.7; and, 5 g TC agar.

2,4-D stock is obtained premade from Phytotech cat# D 295—concentration is 1 mg/ml.

B5 Vitamins Stock (per 100 ml) which is stored in aliquots at −20° C. comprises: 10 g myo-inositol; 100 mg nicotinic acid; 100 mg pyridoxine HCl; and, 1 g thiamine. If the solution does not dissolve quickly enough, apply a low level of heat via the hot stir plate.

Chlorsulfuron Stock comprises 1 mg/ml in 0.01 N Ammonium Hydroxide.

### EXAMPLE 11

### Development of Analytical Methods to detect ZmLox6, Nitrate Reductase, PEP-Carboxylase, and Rubisco

Optimized High Throughput ZmLOX Protein Extraction Technique (From Plant Leaf Tissue)

1. Collect six leaf punches in megatiter tubes, freeze in liquid nitrogen, and place in a mega titer rack.
2. Add 1 stainless steel bead per tube and then add 400 ul of protein extraction buffer.
3. In Genogrinder instrument (Geno/Grinder 2000 from BT&C/OPS Diagnostics, 672 Rt., 202-206 North Bridgewater, N.J., USA), grind the sample at 1×700 setting for 30 s twice. Grind another 30 s if the sample is not completely ground.
4. Centrifuge the megatiter rack at 4000 rpm for 15 min at 4° C.
5. Carefully remove clean supernatant into a 96 well format rack and freeze in liquid nitrogen.

6. To determine the protein concentrations, dilute 10-fold and use BCA™ protein assay kit from Pierce (Pierce Chemical Company, P.O. Box 117, Rockford, Ill., USA).

Extraction buffer

| Reagent | final concentration | | amt. per L | |
|---|---|---|---|---|
| Hepes, pH 7.5 w/KOH | 50 | mM | 11.9 | g |
| Glycerol | 20% | (v/v) | 200 | ml |
| EDTA | 1 | mM | 0.292 | g |
| EGTA | 1 | mM | 0.38 | g |
| Triton X-100 | 0.1% | (v/v) | 10 | ml (10% stock) |
| Benzamidine | 1 | mM | 0.12 | g |
| 6-Aminohexanoic acid | 1 | mM | 0.13 | g |

Add 800 ml RO/di water (to 900 mL)
Adjust pH to 7.5 with ˜5.9 ml 4M KOH solution.
Bring volume to 1 L with RO/di water.

Store prepared buffer at 4° C.

| | final | | | storage |
|---|---|---|---|---|
| Reagent | concentration | | stock sol'n. | location |
| PMSF | 1 | mM | 0.0435 g in 250 ul | desiccator |
| | | | (=1 M)RT | |
| Leupeptin | 10 | uM | 0.00115 g in 250 ul | desiccator |
| | | | (=1 M)10-20° C. | |
| DTT | 1 | mM | 0.154 g | |

Make small aliquots (˜10 ul) and store at −20° C.
* add protease inhibitor frozen stocks to sample aliquot immediately before extraction; see notes

ELISA Procedure for Detection of ZmLox6, Nitrate Reductase, PEP-Carboxylase, and Rubisco
1. Dilute protein from the extraction step is in 25 mM Tris-Cl, pH 9.0, buffer.
2. Aliquot 50 ul of above solution into the wells of a 96-well microtiter plate.
3. Incubate the plate at 37° C. for 2 h or overnight at room temp. No antigen is added to control wells.
4. Rinse the coated plate with de-ionized or distilled water dispensed. Flick the water sticking to the plate and rinse with water two more times, flicking the water from the plate after each rinse.
5. Fill each well with blocking buffer (see below) and incubate 30 min at RT.
6. Repeat step 4.
7. Add 50 ul of the primary antibody solution diluted in blocking buffer to each of the coated wells, wrap plate in plastic wrap, and incubate for 2 h at RT. (1:15,000 dilution of Lox6). No primary antibody is added to the control wells.
8. Rinse plate three times in water as in step 4.
9. Fill each well with blocking buffer and incubate 30 min.
10. Rinse the plate three times with water as step in 4.
11. Add 50 ul secondary antibody solution (1:25,000 dilution of goat anti-rabbit IgG of alkaline phosphatase conjugate antibody; Sigma A3687) in blocking buffer to each of the coated wells, wrap plate in plastic wrap, and incubate for 2 h at RT.
12. Rinse the plate three times in water as step in 4.
13. Fill each well with blocking buffer and incubate 10 min.
14. Rinse plate three times in water as step in 4.
16. Add 75 ul substrate solution to each well and incubate 1 h at room temp in dark.
17. Add 25 ul of 0.5 M NaOH solution to each well to stop the reaction. Mix and measure absorbance at 405 nm.
   10× TBS

0.5 M Tris-Cl, pH 8.0

1.5 M NaCl

Blocking Buffer for One Liter of Solution

100 ml 10× TBS

30 ml 0.3% Triton X100 (10% V/W)

2.5 g BSA

870 ml distilled H₂O

Substrate

Phosphatase substrate 5 mg tablet (Sigma S0942): 1 for 5 ml of buffer.

Substrate Buffer

Diethanolamine 100 g/L

Magnesium Chloride 102 ul of 4.9 M solution.

Thimerosal (sigma T5125) 100 mg

Add all components to 900 ml of deionized water. Adjust the pH to 9.8 with HCl and bring the volume to one liter. Transfer to a sterile 1L bottle and cover with aluminum foil and store at 4° C.

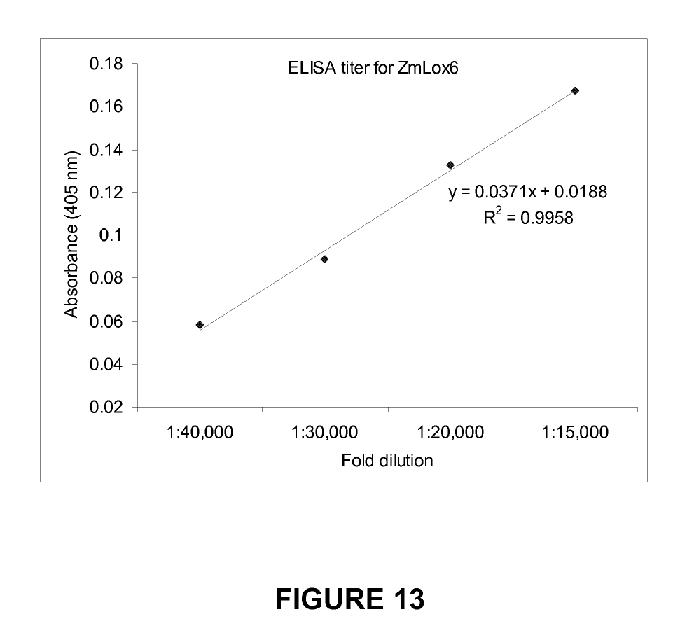

Optimization of Analysis: Optimal protein amount and optimal pH for coating the wells: 50 ul of 10 ug/ml protein at pH 9.0. An example of titrating for antibody dilution is given for the Lox6 protein where the absorbance was linear from 1:15,000 to 1:40,000 dilutions in FIG. 13.

### EXAMPLE 12

### Overexpression of ZmLox6 in Maize Cells Under the Control of Different Promoters

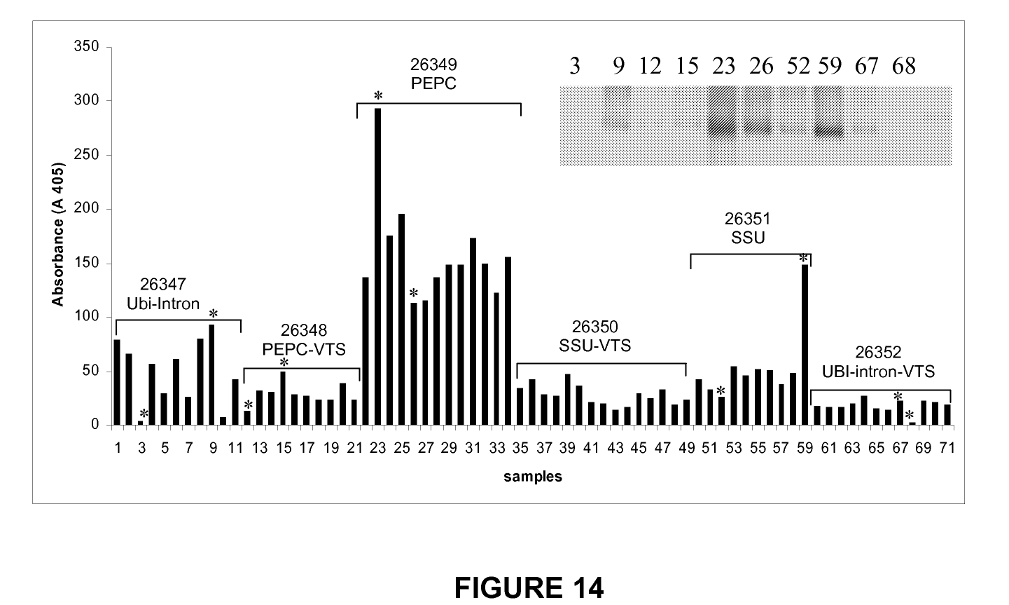

Stable transgenic events of maize were obtained with six different constructs and grown in the greenhouse. Leaf discs were collected as described in the previous examples starting at flowering and then at 10 d or weekly intervals. The ELISA results obtained using the anti-ZmLox6 antibody are shown in FIG. 14. Two main conclusions can be drawn from these results: first, the addition of the vacuolar targeting signal between the promoter and the Lox ORF was detrimental to the expression of its protein and second, maximal expression was obtained with the PEPC promoter, which is specifically expressed in the mesophyll cells. Ubi-Intron promoter gave the next highest expression and Rubisco small subunit the lowest level of expression of the three promoters. On the average, 5-8-fold higher expression of the Lox6 protein was obtained with the PEPC promoter over the wildtype.

### EXAMPLE 13

### Remobilization of the Accumulated Lox6 Protein After Flowering

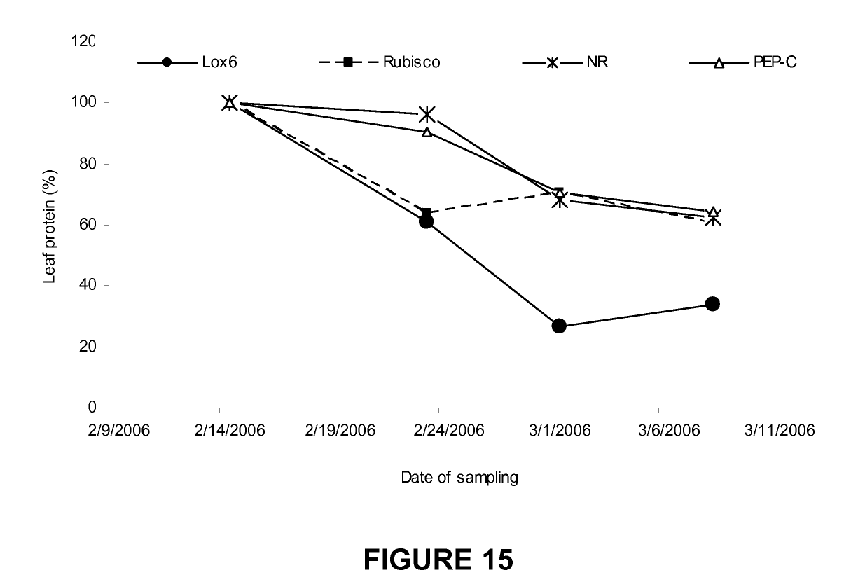

Approximately 80% of the total plant N is accumulated by flowering and 65% of the total N accumulates in the grain at maturity. In other words, a great majority of the N accumulated in the vegetative cells is remobilized to the developing grain. ELISA results from the leaf tissue collected from flowering onwards clearly demonstrate that Lox6 protein is remobilized from the leaves of the To transgenic plants just like the other proteins known to be remobilized, i.e., PEP-carboxylase and Rubisco (FIG. 15).

### EXAMPLE 14

### Accumulation and Remobilization of ZmLox6 Protein in the Field-Grown Plants from the T1 Generation

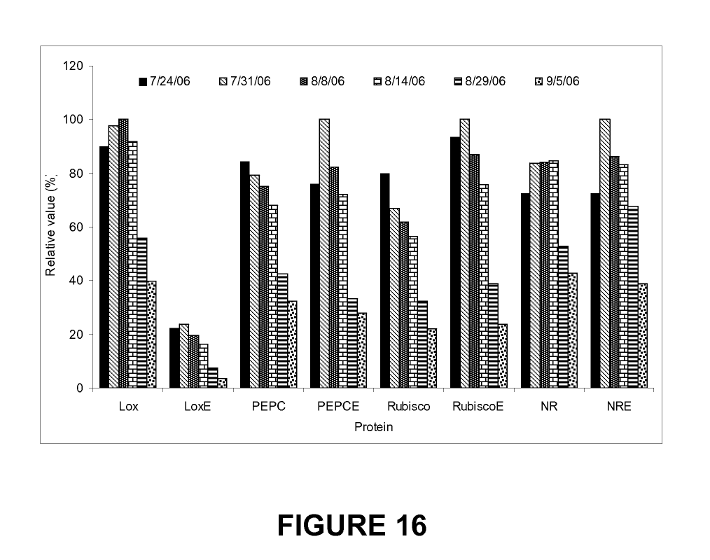

Seed from eight single copy gene insertion events identified by quantitative genomic PCR derived using the PEPC promoter along with the control inbred line was grown in the field in the summer of 2006 in two-row plots. Eight plants were tagged before flowering from each row, 16 plants per event or control. Leaf punches were collected at weekly intervals starting two weeks before flowering and ending two weeks after flowering. When compared to control plants, the Lox6 protein is accumulated at 5-fold higher level than the control events (FIG. 16). The accumulation of the other proteins (PEPC, Rubisco, NR) was not affected to any appreciable extent. The second main conclusion is that the accumulated protein from the transgene is remobilized just as efficiently as the other known proteins, e.g., PEPC and Rubisco (FIG. 16). These results demonstrate that Lox6 protein acts as a vegetative storage protein that is remobilized to the developing grain like the other vegetative proteins.

### EXAMPLE 15

### Variants of LOX Sequences

A. Variant Nucleotide Sequences of LOX Sequences That Do Not Alter the Encoded Amino Acid Sequence

The LOX nucleotide sequence set forth in SEQ ID NO: 1 or 3 is used to generate variant nucleotide sequences having the nucleotide sequence of the open reading frame with about 70%, 76%, 81 %, 86%, 92% and 97% nucleotide sequence identity when compared to the starting unaltered ORF nucleotide sequence of the appropriate SEQ ID NO. These functional variants are generated using a standard codon table. While the nucleotide sequence of the variant is altered, the amino acid sequence encoded by the open reading frame does not change.

B. Variant Amino Acid Sequences of a LOX6 Sequence

Variant amino acid sequences of LOX6 sequence are generated. In this example, one amino acid is altered. Specifically, the open reading frame set forth in SEQ ID NO: 3, or SEQ ID NO: 1 (at 62-2737) is reviewed to determined the appropriate amino acid alteration. The selection of the amino acid to change is made by consulting the protein alignment (with the other orthologs and other gene family members from various species). See FIG. 7 and Table 2. An amino acid is selected that is deemed not to be under high selection pressure (not highly conserved) and which is rather easily substituted by an amino acid with similar chemical characteristics (i.e., similar functional side-chain). Using the protein alignment set forth in FIG. 7, and Table 2, an appropriate amino acid can be changed. Once the targeted amino acid is identified, the procedure outlined in Example 6A is followed. Variants having about 70%, 75%, 81%, 86%, 92% and 97% nucleic acid sequence identity to SEQ ID NO: 1 or 3 are generated using this method.

C. Additional Variant Amino Acid Sequences of LOX6 Sequences

In this example, artificial protein sequences are created having 82%, 87%, 92% and 97% identity relative to the reference protein sequence. This latter effort requires identifying conserved and variable regions from the alignment set forth in FIG. 7 and then the judicious application of an amino acid substitutions table. These parts will be discussed in more detail below.

Largely, the determination of which amino acid sequences are altered is made based on the conserved regions among LOX6 protein or among the other LOX proteins. See FIG. 7. Based on the sequence alignment, the various regions of the LOX sequences that can likely be altered are represented in lower case letters, while the conserved regions are represented by capital letters. It is recognized that conservative substitutions can be made in the conserved regions below without altering function. In addition, one of skill will understand that functional variants of the LOX sequence of the invention can have minor non-conserved amino acid alterations in the conserved domain.

Artificial protein sequences are then created that are different from the original in the intervals of 80-85%, 85-90%, 90-95% and 95-100% identity. Midpoints of these intervals are targeted, with liberal latitude of plus or minus 1 %, for example. The amino acids substitutions will be effected by a custom Perl script. The substitution table is provided below in Table 2.

TABLE 2
Substitution Table

| | Strongly Similar | Rank of | |
|---|---|---|---|
| Amino | and Optimal | Order | |
| Acid | Substitution | to Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine |
| | | | cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C, and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involved a 50:50 substitution of the two alternate optimal substitutions.

The variant amino acid sequences are written as output. Perl script is used to calculate the percent identities. Using this procedure, variants of LOX sequences are generating having about 82%, 87%, 92% and 97% amino acid identity to the starting unaltered ORF nucleotide sequence of the corresponding SEQ ID NO.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

That which is claimed is:

1. A method for increasing the nitrogen storage capacity of a plant, said method comprising introducing into said plant at least one nucleotide construct comprising a nucleotide sequence operably linked to a promoter that drives expression in a plant cell, wherein said nucleotide sequence is selected from the group consisting of:
(a) a nucleotide sequence comprising the sequence set forth in SEQ ID NO: 1, the sequence set forth in nucleotides 62-2737 of SEQ ID NO: 1, or the sequence set forth in SEQ ID NO: 3;
(b) a nucleotide sequence encoding the amino acid sequence set forth in SEQ ID NO: 2;
(c) a nucleotide sequence having at least 90% sequence identity to the sequence set forth in SEQ ID NO: 1, the sequence set forth in nucleotides 62-2737 of SEQ ID NO: 1, or the sequence set forth in SEQ ID NO: 3, wherein said nucleotide sequence encodes a polypeptide having vegetative storage protein properties;
(d) a nucleotide sequence that hybridizes under stringent conditions to the complement of the nucleotide sequence of (a) or (b), wherein said stringent conditions comprise hybridization in 50% formamide, 1 M NaCl, 1% SDS at 37° C., and a wash in 0.1×SSC at 60° C. to 65° C., wherein said nucleotide sequence encodes a polypeptide having vegetative storage protein properties; and
(e) a nucleotide sequence encoding an amino acid sequence having at least 90% sequence identity to the sequence set forth in SEQ ID NO: 2, wherein said polynucleotide encodes a polypeptide having vegetative storage protein properties.

2. The method of claim 1, wherein said promoter is a tissue-preferred promoter.

3. The method of claim 2, wherein said tissue-preferred promoter is a leaf-preferred promoter.

4. The method of claim 3, wherein said plant is a C4 plant.

5. The method of claim 4, wherein said promoter is a mesophyll cell-preferred promoter.

6. The method of claim 5, wherein said nucleotide construct comprises a coding sequence for a vacuolar sorting signal operably linked to said nucleotide sequence.

7. The method of claim 5, wherein said nucleotide construct comprises a coding sequence for a plastid transit peptide operably linked to said nucleotide sequence.

8. The method of claim 4, wherein said promoter is a bundle-sheath cell-preferred promoter.

9. The method of claim 8, wherein said nucleotide construct comprises a coding sequence for a vacuolar sorting signal operably linked to said nucleotide sequence.

10. The method of any one of claims 4 to 9, wherein said C4 plant is maize, sorghum, or sugarcane.

11. The method of claim 1, wherein said promoter is a constitutive promoter.

12. The method of claim 1, wherein said promoter is an inducible promoter.

13. The method of claim 12, wherein said inducible promoter is a wound-inducible promoter.

14. The method of any one of claims 1 to 3 and 11 to 13, wherein said plant is a monocot.

15. The method of claim 14, wherein said monocot is selected from the group consisting of maize, wheat, rice, barley, sorghum, or rye.

16. The method of claim 1, wherein said nucleotide construct comprises a coding sequence for a vacuolar sorting signal operably linked to said nucleotide sequence.

17. The method of claim 16, wherein said promoter is a tissue-preferred promoter.

18. The method of claim 17, wherein said tissue-preferred promoter is a leaf-preferred promoter.

19. The method of claim 16, wherein said promoter is a constitutive promoter.

20. The method of claim 16, wherein said promoter is an inducible promoter.

21. The method of claim 20, wherein said inducible promoter is a wound-inducible promoter.

22. The method of any one of claims 16 to 21, wherein said plant is a monocot.

23. The method of claim 22, wherein said monocot is selected from the group consisting of maize, wheat, rice, barley, sorghum, or rye.

24. A method for increasing the nutritional value of forage or silage, said method comprising introducing into a plant used for forage or silage at least one nucleotide construct comprising a nucleotide sequence operably linked to a promoter that drives expression in a plant cell, wherein said nucleotide sequence is selected from the group consisting of:
(a) a nucleotide sequence comprising the sequence set forth in SEQ ID NO: 1, the sequence set forth in nucleotides 62-2737 of SEQ ID NO: 1, or the sequence set forth in SEQ ID NO: 3;
(b) a nucleotide sequence encoding the amino acid sequence set forth in SEQ ID NO:2;
(c) a nucleotide sequence having at least 90% sequence identity to the sequence set forth in SEQ ID NO: 1, the sequence set forth in nucleotides 62-2737 of SEQ ID NO: 1, or the sequence set forth in SEQ ID NO: 3, wherein said nucleotide sequence encodes a polypeptide having vegetative storage protein properties;
(d) a nucleotide sequence that hybridizes under stringent conditions to the complement of the nucleotide sequence of (a) or (b), wherein said stringent conditions comprise hybridization in 50% formamide, 1 M NaCl, 1% SDS at 37° C., and a wash in 0.1×SSC at 60° C. to 65° C., wherein said nucleotide sequence encodes a polypeptide having vegetative storage protein properties; and
(e) a nucleotide sequence encoding an amino acid sequence having at least 90% sequence identity to the sequence set forth in SEQ ID NO:2, wherein said polynucleotide encodes a polypeptide having vegetative storage protein properties.

25. The method of claim 24, wherein said nucleotide sequence of (e) encodes a vegetative storage protein that is enriched in essential amino acids.

26. The method of claim 25, wherein said essential amino acids include one or more amino acids selected from the group consisting of lysine, methionine, tryptophan, threonine, phenylalanine, leucine, valine, and isoleucine.

27. The method of any one of claims 24 to 26, wherein said promoter is a tissue-preferred promoter.

28. The method of claim 27, wherein said tissue-preferred promoter is a leaf-preferred promoter.

29. The method of claim 28, wherein said plant is a C4 plant.

30. The method of claim 29, wherein said promoter is a mesophyll cell-preferred promoter.

31. The method of claim 30, wherein said nucleotide construct comprises a coding sequence for a vacuolar sorting signal operably linked to said nucleotide sequence.

32. The method of claim 30, wherein said nucleotide construct comprises a coding sequence for a plastid transit peptide operably linked to said nucleotide sequence.

33. The method of claim 29, wherein said promoter is a bundle-sheath cell-preferred promoter.

34. The method of claim 33, wherein said nucleotide construct comprises a coding sequence for a vacuolar sorting signal operably linked to said nucleotide sequence.

35. The method of any one of claims 29 to 34, wherein said C4 plant is maize, sorghum, or sugarcane.

36. The method of any one of claims 24 to 26, wherein said promoter is a constitutive promoter.

37. The method of any one of claims 24 to 26, wherein said promoter is an inducible promoter.

38. The method of claim 37, wherein said inducible promoter is a wound-inducible promoter.

39. The method of any one of claims 24 to 28 and 36 to 38, wherein said plant is a monocot.

40. The method of claim 39, wherein said monocot is selected from the group consisting of maize, wheat, rice, barley, sorghum, or rye.

41. The method of any one of claims 24 to 26, wherein said nucleotide construct comprises a coding sequence for a vacuolar sorting signal operably linked to said nucleotide sequence.

42. The method of claim 41, wherein said promoter is a tissue-preferred promoter.

43. The method of claim 42, wherein said tissue-preferred promoter is a leaf-preferred promoter.

44. The method of claim 41, wherein said promoter is a constitutive promoter.

45. The method of claim 41, wherein said promoter is an inducible promoter.

46. The method of claim 45, wherein said inducible promoter is a wound-inducible promoter.

47. The method of anyone of claims 41 to 46, wherein said plant is a monocot.

48. The method of claim 47, wherein said monocot is selected from the group consisting of maize, wheat, rice, barley, sorghum, or rye.

49. A method for increasing the nitrogen content in a plant or plant part thereof, said method comprising introducing into said plant at least one nucleotide construct comprising a nucleotide sequence operably linked to a promoter that drives expression in a plant cell, wherein said nucleotide sequence is selected from the group consisting of:
(a) a nucleotide sequence comprising the sequence set forth in SEQ ID NO: 1, the sequence set forth in nucleotides 62-2737 of SEQ ID NO: 1, or the sequence set forth in SEQ ID NO: 3;
(b) a nucleotide sequence encoding the amino acid sequence set forth in SEQ ID NO: 2;
(c) a nucleotide sequence having at least 90% sequence identity to the sequence set forth in SEQ ID NO: 1, the sequence set forth in nucleotides 62-2737 of SEQ ID NO: 1, or the sequence set forth in SEQ ID NO: 3, wherein said nucleotide sequence encodes a polypeptide having vegetative storage protein properties;
(d) a nucleotide sequence that hybridizes under stringent conditions to the complement of the nucleotide sequence of (a) or (b), wherein said stringent conditions comprise hybridization in 50% formamide, 1 M NaCl, 1% SDS at 37° C., and a wash in 0.1×SSC at 60° C. to 65° C., wherein said nucleotide sequence encodes a polypeptide having vegetative storage protein properties; and
(e) a nucleotide sequence encoding an amino acid sequence having at least 90% sequence identity to the sequence set forth in SEQ ID NO: 2, wherein said polynucleotide encodes a polypeptide having vegetative storage protein properties.

50. The method of claim 49, wherein said nucleotide sequence of (e) encodes a vegetative storage protein that is enriched in essential amino acids.

51. The method of claim 50, wherein said essential amino acids include one or more amino acids selected from the group consisting of lysine, methionine, tryptophan, threonine, phenylalanine, leucine, valine, and isoleucine.

52. The method of any one of claims 49 to 51, wherein said plant or plant part is used for forage or silage.

53. The method of claim 52, wherein said plant part used for forage or silage is selected from the group consisting of leaves, stems, seeds, and any combination thereof.

54. The method of any one of claims 49 to 53, wherein said promoter is a tissue-preferred promoter.

55. The method of claim 54, wherein said tissue-preferred promoter is a leaf-preferred promoter.

56. The method of claim 55, wherein said plant is a C4 plant.

57. The method of claim 56, wherein said promoter is a mesophyll cell-preferred promoter.

58. The method of claim 57, wherein said nucleotide construct comprises a coding sequence for a vacuolar sorting signal operably linked to said nucleotide sequence.

59. The method of claim 57, wherein said nucleotide construct comprises a coding sequence for a plastid transit peptide operably linked to said nucleotide sequence.

60. The method of claim 56, wherein said promoter is a bundle-sheath cell-preferred promoter.

61. The method of claim 60, wherein said nucleotide construct comprises a coding sequence for a vacuolar sorting signal operably linked to said nucleotide sequence.

62. The method of any one of claims 56 to 61, wherein said C4 plant is maize, sorghum, or sugarcane.

63. The method of any one of claims 49 to 53, wherein said promoter is a constitutive promoter.

64. The method of any one of claims 49 to 53, wherein said promoter is an inducible promoter.

65. The method of claim 64, wherein said inducible promoter is a wound-inducible promoter.

66. The method of any one of claims 49 to 55 and 63 to 65, wherein said plant is a monocot.

67. The method of claim 66, wherein said monocot is selected from the group consisting of maize, wheat, rice, barley, sorghum, or rye.

68. The method of any one of claims 49 to 53, wherein said nucleotide construct comprises a coding sequence for a vacuolar sorting signal operably linked to said nucleotide sequence.

69. The method of claim 68, wherein said promoter is a tissue-preferred promoter.

70. The method of claim 69, wherein said tissue-preferred promoter is a leaf-preferred promoter.

71. The method of claim 68, wherein said promoter is a constitutive promoter.

72. The method of claim 68, wherein said promoter is an inducible promoter.

73. The method of claim 72, wherein said inducible promoter is a wound-inducible promoter.

74. The method of any one of claims 68 to 73, wherein said plant is a monocot.

75. The method of claim 74, wherein said monocot is selected from the group consisting of maize, wheat, rice, barley, sorghum, or rye.

76. A nucleotide construct comprising a coding sequence for a vacuolar sorting signal and a nucleotide sequence encoding a polypeptide having vegetative storage protein properties, wherein said coding sequence and said nucleotide sequence are operably linked to a promoter that drives expression in a plant cell, and wherein said nucleotide sequence is selected from the group consisting of:
(a) a nucleotide sequence comprising the sequence set forth in SEQ ID NO: 1, the sequence set forth in nucleotides 62-2737 of SEQ ID NO: 1, or the sequence set forth in SEQ ID NO: 3;
(b) a nucleotide sequence encoding the amino acid sequence set forth in SEQ ID NO: 2;
(c) a nucleotide sequence having at least 90% sequence identity to the sequence set forth in SEQ ID NO: 1, the sequence set forth in nucleotides 62-2737 of SEQ ID NO: 1, or the sequence set forth in SEQ ID NO: 3;
(d) a nucleotide sequence that hybridizes under stringent conditions to the complement of the nucleotide sequence of (a) or (b), wherein said stringent conditions comprise hybridization in 50% formamide, 1 M NaCl, 1% SDS at 37° C., and a wash in 0.1×SSC at 60° C. to 65° C.; and
(e) a nucleotide sequence encoding an amino acid sequence having at least 90% sequence identity to the sequence set forth in SEQ ID NO: 2.

77. The nucleotide construct of claim 76, wherein said nucleotide sequence of (e) encodes a vegetative storage protein that is enriched in essential amino acids.

78. The nucleotide construct of claim 77, wherein said essential amino acids include one or more amino acids selected from the group consisting of lysine, methionine, tryptophan, threonine, phenylalanine, leucine, valine, and isoleucine.

79. The nucleotide construct of any one of claims 76 to 78, wherein said promoter is a tissue-preferred promoter.

80. The nucleotide construct of claim 79, wherein said tissue-preferred promoter is a leaf-preferred promoter.

81. The nucleotide construct of any one of claims 76 to 78, wherein said promoter is a constitutive promoter.

82. The nucleotide construct of any one of claims 76 to 78, wherein said promoter is an inducible promoter.

83. The nucleotide construct of claim 82, wherein said inducible promoter is a wound-inducible promoter.

84. A plant comprising the nucleotide construct of any one of claims 76 to 83.

85. The plant of claim 84, wherein said plant is a monocot.

86. The plant of claim 85, wherein said monocot is maize, wheat, rice, barley, sorghum, or rye.

87. The plant of any one of claims 84 to 86, wherein said nucleotide construct is stably incorporated into the genome of said plant.

88. A transgenic seed of the plant of any one of claims 84 to 87.

89. A method of determining the ZmLox expression in a plant tissue comprising:
a. harvesting plant tissue from chosen plants;
b. extracting plant protein using optimized high throughput ZmLOX protein extraction technique; and
c. analyzing said extracted protein by ELISA to determine level of ZmLox expression.
